(19) United States
(12) Patent Application Publication
(10) Pub. No.: US 2020/0082947 A1
(43) Pub. Date: Mar. 12, 2020

(54) Methods and Systems for Pedigree Enrichment and Family-Based Analyses Within Pedigrees

(71) Applicant: Regeneron Pharmaceuticals, Inc., Tarrytown, NY (US)

(72) Inventors: Jeffrey Staples, Tarrytown, NY (US); Claudia Gonzaga-Jauregui, Tarrytown, NY (US); Jeffrey Reid, Tarrytown, NY (US); Lukas Habegger, Tarrytown, NY (US)

(21) Appl. No.: 16/563,222

(22) Filed: Sep. 6, 2019

Related U.S. Application Data
(60) Provisional application No. 62/728,536, filed on Sep. 7, 2018.

(51) Int. Cl. G16H 50/80; G16H 10/60; G06N 3/12

(52) U.S. Cl. CPC G16H 50/80; G06N 3/126; G16H 10/60

(57) ABSTRACT

Methods, non-transitory computer-implemented methods and systems for creating enriched pedigree are provided. Also provided are methods, non-transitory computer-implemented methods and systems for determining a disease-causing variant.

### CROSS-REFERENCE TO RELATED APPLICATION

This application claims the benefit of U.S. Provisional Patent Application No. 62/728,536, filed on Sep. 7, 2018; the content of this application is hereby incorporated by reference in its entirety.

### FIELD

This disclosure relates generally to methods and systems for pedigree enrichment in a large population cohort. More particularly, the disclosure relates to systems and methods for identifying affecteds in first-degree family networks to enrich pedigrees using sequencing data and further identifying variant-trait pairs that co-segregate within pedigrees and across pedigrees to connect rare genetic variations to disease and disease susceptibility.

### BACKGROUND

Clinical investigators are continually seeking to identify pathogenic variants responsible for diseases. Cytogenomic arrays and genotyping of linkage panels remain useful approaches for the identification of copy number variation and for identifying co-segregating haplotypes within large Mendelian (especially dominant) disease families, respectively. However, optimal approaches to discovering pathogenic variants in complex diseases remain unclear.

Following transmission of variants through a genealogy is at the foundation of modern genetics. Most genetic disorders are heterogeneous with a range of a few genes to many genes playing a role in causing disease. The genetic defect in a number of rare disorders remains elusive. With the classical positional cloning technique, a substantial number of affected families are required to identify the region in which the causative gene should reside, and for rare disorders, these families are not always available. Moreover, identifying a region of interest is not sufficient; the genes within this region all have to be sequenced, which can be quite laborious. With the advent of next-generation sequencing, whole genomes or exomes of patients without the need to select a candidate genetic region can be studied. Although we can now discover and genotype rare genetic variants in large study cohorts, the majority of these variants will be present in only a few individuals—in population-based genetic studies, >50% of variants are seen in a single individual—making it difficult to establish evidence of association.

It is further particularly challenging to investigate the impact that rare variants have on these heterogeneous disorders in genome-wide scans of large genetic cohorts. Unambiguous assignment of disease causality for sequence variants is often impossible, particularly for the very low-frequency variants underlying many cases of rare, severe diseases. However, if a set of related individuals that share a given genetic disorder are identified, then this heterogeneity is greatly reduced, allowing focusing on single genes and variants driving a specific phenotype segregating in the affected individuals within a pedigree.

The potential of genome-wide association studies (GWAS) to enable an unbiased search for disease loci across the entire human genome provides an unprecedented research opportunity in genetics. Interrogating several hundred thousand single nucleotide polymorphisms (SNPs) across many subjects at the same time raises many statistical challenges in the design and analysis of these studies. Genotyping on such a scale requires new methodology for handling data quality issues; likewise, association tests are computed for hundreds of thousands of markers, whose results have to be adjusted for multiple comparisons. The magnitude of these problems raises the question of whether the new technical ability to genotype such dense SNP sets will translate into the identification of novel genetic disease loci or whether the technical advance remains under-utilized. There are at least two ways to approach such of genome-wide association studies—population-based and family-based designs.

Population-based studies have a sample size of several thousand subjects (Szklo M. Epidemiologic Reviews (1998) 20 (1): 81-90). However, these studies are expensive, time consuming, and can encounter phenotypic and genotypic heterogeneity due to the large sample size (Sorlie and Wei. Journal of American College of Cardiology (2011) 58(19): 2010-3; Laird and Lange. Statistical Science (2009) 24(4): 388-397).

Family-based analyses can be particularly informative when interrogating rare variants of potential moderate-to-large effects co-segregating with a phenotype of interest, and these variants may not be easily detected with a population-based analysis. A key benefit of family-based association studies is the control for confounding bias due to population stratification, albeit at a potential loss of power (Witte et al. American Journal of epidemiology (1999) 149(8): 693-705; Thomas et al. Cancer (2003) 97(8): 1894-1903).

There are many large-scale sequencing initiatives for ascertaining and sequencing hundreds of thousands of de-identified individuals, such as, DiscovEHR, UK Biobank, the US government's All of US (part of the Precision Medicine Initiative), TOPMed, ExAC/gnomAD, and many others (Dewey et al. Science (2016); 254, aaf6814; Sudlow et al. PLoSMed. (2015) 12, e1001779; Collins et al. (2016) New England Journal of Medicine (2015) 372, 793-795; Lek et al. Nature (2016) 536, 285-291). Pedigrees can be constructed from such large datasets of protein sequencing information, which can be used by investigators to determine the heritability and genetic models for traits and disorders. Knowing the exact pedigree structure allows to correctly identify the genetic mode of disease inheritance and utilize powerful genetic-analysis tools that require, or benefit from, the true pedigree structure. However, there exists a challenge to directly obtain accurate pedigree records from de-identified health records, precluding many powerful family-based analyses.

A close pairwise relationships can be used for reconstructing pedigree structures directly from the genetic data with tools such as PRIMUS and CLAPPER (Staples et al. American Journal of Human Genetics (2014) 95, 553-564 and Ko and Nielson. PLoS Genet. (2017) 13, e1006963). Although estimated relationships and pedigrees are extremely useful, there exists a concern regarding the use of estimated relationships and pedigrees with significant statistical uncertainty in analyses that are sensitive to inaccuracies in estimated relationships and pedigree structures.

While precision medicine cohorts may not readily have pedigree information, informative pedigrees can be obtained directly from the genetic data to create a large cohort for traditional Mendelian analyses. Identifying pedigrees that are enriched for affecteds with phenotypes of interest can be used in an effort to identify the causal (rare) variation driving these phenotypes, since the genetic cause is more likely to be shared within a family unit. Defining the sets of affected individuals used in the pedigree enrichment analysis can be critical. Thus, there is a need for such methods or systems to allow pedigree enrichment. These enriched pedigrees can be leveraged to help define subsets of related participants with phenotypes of interest and then examine these subsets to identify genetic drivers of traits and disease. There remains a need for improved bioinformatics tools for pedigree enrichment to identify potentially informative pedigree-phenotype pairings that enable traditional Mendelian analyses at a large scale.

The discovery of methods and systems to generated enriched pedigrees can guide drug discovery scientists to understand critical roles played by certain proteins and their variants in normal physiology or in the causation of disease and to elucidate their function both biochemically and biologically (Lele R. J. Assoc. Physicians India (2003) 51: 373-380).

The methods and systems described herein will provide an enriched pedigree which can lead to identifying such diseases-causing variant(s) and thus fuel drug discovery efforts and clinical investigation efforts.

### SUMMARY

In one exemplary aspect, the disclosure provides methods for generating an enriched pedigree by generating a first degree network of individuals based on sequencing data of a cohort, identifying individuals in the cohort as an affected or an unaffected and creating the enriched pedigree containing the affecteds and the unaffecteds.

In some exemplary embodiments, the method for generating an enriched pedigree can comprise identifying individuals in a pedigree as an affected or an unaffected, wherein the individual with at least one binary trait is identified as affected and the individual without the at least one binary trait is identified as unaffected, and then evaluating whether the pattern of affected and unaffected individuals is consistent with a Mendelian mode of inheritance (e.g., autosomal dominant, autosomal recessive, x-linked dominant, x-linked recessive, or y-linked). In some specific exemplary embodiments, the binary trait can be defined using the International Statistical Classification of Diseases and Related Health Problems (ICD), a medical classification list by the World Health Organization (WHO) which contains codes for diseases, signs and symptoms, abnormal findings, complaints, social circumstances, and external causes of injury or diseases. The ninth or the tenth version of the ICD can be used to define the binary traits. In one exemplary embodiment, the individual for which no electronic health record data can be available for the specific binary trait, or who has conflicting or unreliable data for the specific binary trait, irrespective of the absence or presence of the specific binary trait in the medical record, can be determined to be an unknown affected.

In some exemplary embodiments, the method for generating an enriched pedigree can comprise identifying individuals in a pedigree as an affected or an unaffected, wherein the individual with at least one extreme quantitative trait is identified as affected and the individual without the at least one extreme quantitative trait is identified as unaffected, and then evaluating whether the pattern of affected and unaffected individuals is consistent with either a Mendelian mode of inheritance (e.g., autosomal dominant, autosomal recessive, x-linked dominant, x-linked recessive, or y-linked). Several parameters can be used to define whether or not someone is affected by an extreme quantitative trait, such as a maximum age cutoff to define an earlier onset of disorder, or having minimum or maximum or median measurement of a quantitative trait exceeded a defined statistical cutoff of deviation from normal population measurement of the trait (e.g., 2 standard deviations above the population mean). In one exemplary embodiment, the individual for which no electronic health record data can be available for the specific quantitative trait or who has conflicting or unreliable data for the specific quantitative trait, irrespective of the absence or presence of the specific quantitative trait in the medical record, can be determined to be an unknown affected.

In some exemplary embodiments, the method for generating an enriched pedigree can comprise identifying individuals in a pedigree as an affected or an unaffected, wherein the individual with at least one binary trait, extreme quantitative trait, or combination thereof is identified as affected and the individual without the at least one binary trait, extreme quantitative trait, or combination thereof is identified as unaffected. The binary trait can be a defined ICD code as described above. Several parameters can be used to define extreme quantitative traits as described above. In one exemplary embodiment, the individual for whom no electronic health record data is available for the specific binary trait, quantitative trait, or combination thereof or who has conflicting or unreliable data for the specific binary trait, quantitative trait, or combination thereof, irrespective of the absence or presence of the specific quantitative trait in the medical record, can be determined to be an unknown affected.

In some exemplary embodiments, the method for generating an enriched pedigree can comprise identifying individuals in a pedigree as an affected or an unaffected, wherein the individual with at least one binary trait, extreme quantitative trait, or combination thereof is identified as affected and the individual without the at least one binary trait, extreme quantitative trait, or combination thereof is identified as unaffected, and wherein the at least one binary trait, an extreme quantitative trait, or combination thereof can include two or more similar or complementary traits.

In some exemplary embodiments, the method for generating an enriched pedigree can comprise identifying individuals in a pedigree as an affected or an unaffected, wherein the individual with at least one binary trait, extreme quantitative trait, or combination thereof is identified as affected and the individual without the at least one binary trait, extreme quantitative trait, or combination thereof is identified as unaffected, and wherein the at least one binary trait, an extreme quantitative trait, or combination thereof can include taking an intersection of two or more extreme or interesting traits.

In some exemplary embodiments, the method for generating an enriched pedigree can comprise identifying individuals in a pedigree as an affected, wherein the individual with at least one binary trait, extreme quantitative trait, or combination thereof is identified as affected and defining the individual determined to be affected as affected carrier of an association result from external analyses.

In some exemplary embodiments, the method for generating an enriched pedigree comprises generating a first degree network of individuals based on sequencing data of a cohort. The sequencing data can include whole genome sequencing data, exome sequencing data, or genotype data.

In some exemplary embodiments, the method for generating an enriched pedigree comprises generating a first degree network of individuals based on exome sequencing data. The first degree network of individuals based on exome sequencing data can be generated by leveraging the population's relatedness including: removing low-quality sequence variants from a dataset of nucleic acid sequence samples obtained from a plurality of human subjects, establishing an ancestral superclass designation for each of one or more of the samples, removing low-quality samples from the dataset, generating first identity-by-descent estimates of subjects within an ancestral superclass, generating second identity-by-descent estimates of subjects independent from subjects' ancestral superclass, and clustering subjects into primary first-degree family networks based on one or more of the second identity-by-descent estimates.

In some exemplary embodiments, the method for generating an enriched pedigree comprises generating a first degree network of individuals based on sequencing data of a cohort wherein the cohort can include any dataset comprising a plurality of subjects.

In some exemplary embodiments, the method for creating the enriched pedigree further includes enriching the pedigree based on a p-value. The enrichment can include defining a “founder anchored branch” or “branch” of a pedigree as all descendants of a founder within a pedigree and using a binomial test to evaluate if the branch is enriched for a binary trait. The binary trait could be defined using the ICD as described above. The enrichment can also include defining a “founder anchored branch” or “branch” of a pedigree as all descendants of a founder within a pedigree and using a t-test to evaluate if the branch if enriched for an extreme quantitative trait. Several parameters can be used to define extreme quantitative traits as described above. Further, the enrichment can also include applying a multiple-test p-value cutoff.

In one exemplary aspect, the disclosure provides methods for identifying a disease-causing variant by generating an enriched pedigree by generating a first degree network of individuals based on sequencing data of a cohort, identifying individuals in the cohort as an affected or an unaffected, creating at least one enriched pedigree containing the affecteds and the unaffecteds, performing segregation analysis to identify variant trait pairs that co-segregate within and across at least one enriched pedigree and analyzing the variant trait pairs to identify the disease-causing variant.

In some exemplary embodiments, the method for identifying a disease-causing variant can comprise identifying individuals in a pedigree as an affected or an unaffected, wherein the individual with at least one binary trait is identified as affected and the individual without the at least one binary trait is identified as unaffected, and then evaluating whether the pattern of affected and unaffected individuals is consistent with a Mendelian mode of inheritance (e.g., autosomal dominant, autosomal recessive, x-linked dominant, x-linked recessive, or y-linked). In some specific exemplary embodiments, the binary trait can be defined using the International Statistical Classification of Diseases and Related Health Problems (ICD), a medical classification list by the World Health Organization (WHO) which contains codes for diseases, signs and symptoms, abnormal findings, complaints, social circumstances, and external causes of injury or diseases. The ninth or the tenth version of the ICD can be used to define the binary traits. In one exemplary embodiment, the individual for which no electronic health record data can be available for the specific binary trait, or who has conflicting or unreliable data for the specific binary trait, irrespective of the absence or presence of the specific binary trait in the medical record, can be determined to be an unknown affected.

In some exemplary embodiments, the method for identifying a disease-causing variant can comprise identifying individuals in a pedigree as an affected or an unaffected, wherein the individual with at least one extreme quantitative trait is identified as affected and the individual without the at least one extreme quantitative trait is identified as unaffected, and then evaluating whether the pattern of affected and unaffected individuals is consistent with either a Mendelian mode of inheritance (e.g., autosomal dominant, autosomal recessive, x-linked dominant, x-linked recessive, or y-linked). Several parameters can be used to define whether or not someone is affected by an extreme quantitative trait, such as a maximum age cutoff to define an earlier onset of disorder, or having minimum or maximum or median measurement of the quantitative trait exceeded a defined statistical cutoff of deviation from normal population measurement of the trait (e.g., 2 standard deviations above the population mean). In one exemplary embodiment, the individual for which no electronic health record data can be available for the specific quantitative trait or who has conflicting or unreliable data for the specific quantitative trait, irrespective of the absence or presence of the specific quantitative trait in the medical record, can be determined to be an unknown affected.

In some exemplary embodiments, the method for identifying a disease-causing variant can comprise identifying individuals in a pedigree as an affected or an unaffected, wherein the individual with at least one binary trait, extreme quantitative trait, or combination thereof is identified as affected and the individual without the at least one binary trait, extreme quantitative trait, or combination thereof is identified as unaffected. The binary trait can be a defined ICD code as described above. Several parameters can be used to define extreme quantitative traits as described above. In one exemplary embodiment, the individual for whom no electronic health record data is available for the specific binary trait, quantitative trait, or combination thereof or who has conflicting or unreliable data for the specific binary trait, quantitative trait, or combination thereof, irrespective of the absence or presence of the specific quantitative trait in the medical record, can be determined to be an unknown affected.

In some exemplary embodiments, the method for identifying a disease-causing variant can comprise identifying individuals in a pedigree as an affected or an unaffected, wherein the individual with at least one binary trait, extreme quantitative trait, or combination thereof is identified as affected and the individual without the at least one binary trait, extreme quantitative trait, or combination thereof is identified as unaffected, and wherein the at least one binary trait, an extreme quantitative trait, or combination thereof can include two or more similar or complementary traits.

In some exemplary embodiments, the method for identifying a disease-causing variant can comprise identifying individuals in a pedigree as an affected or an unaffected, wherein the individual with at least one binary trait, extreme quantitative trait, or combination thereof is identified as affected and the individual without the at least one binary trait, extreme quantitative trait, or combination thereof is identified as unaffected, and wherein the at least one binary trait, an extreme quantitative trait, or combination thereof can include taking an intersection of two or more extreme or interesting traits.

In some exemplary embodiments, the method for identifying a disease-causing variant can comprise identifying individuals in a pedigree as an affected, wherein the individual with at least one binary trait, extreme quantitative trait, or combination thereof is identified as affected and defining the individual determined to be affected and defining the individual determined to be affected as affected carrier of an association result from external analyses.

In some exemplary embodiments, the method for identifying a disease-causing variant comprises generating a first degree network of individuals based on sequencing data of a cohort. The sequencing data can include whole genome sequencing data, exome sequencing data, or genotype data.

In some exemplary embodiments, the method for identifying a disease-causing variant comprises generating a first degree network of individuals based on exome sequencing data. The first degree network of individuals based on exome sequencing data can be generated by leveraging the population's relatedness including: removing low-quality sequence variants from a dataset of nucleic acid sequence samples obtained from a plurality of human subjects, establishing an ancestral superclass designation for each of one or more of the samples, removing low-quality samples from the dataset, generating first identity-by-descent estimates of subjects within an ancestral superclass, generating second identity-by-descent estimates of subjects independent from subjects' ancestral superclass, and clustering subjects into primary first-degree family networks based on one or more of the second identity-by-descent estimates.

In some exemplary embodiments, the method for identifying a disease-causing variant comprises generating a first degree network of individuals based on sequencing data of a cohort wherein the cohort can include any dataset comprising a plurality of subjects.

In some exemplary embodiments, the method for creating the enriched pedigree further includes enriching the pedigree based on a p-value. The enrichment can include defining a “founder anchored branch” or “branch” of a pedigree as all descendants of a founder within a pedigree and using a binomial test to evaluate if the branch is enriched for a binary trait. The binary trait could be defined using the ICD as described above. The enrichment can also include defining a “founder anchored branch” or “branch” of a pedigree as all descendants of a founder within a pedigree and using a t-test to evaluate if the branch if enriched for an extreme quantitative trait. Several parameters can be used to define extreme quantitative traits as described above. Further, the enrichment can also include applying a multiple-test p-value cutoff.

In some exemplary embodiments, the method for identifying a disease-causing variant can comprise identifying variant trait pairs that co-segregate with affecteds within the pedigree, and performing a segregation analysis which includes finding at least one enriched pedigree based on phenotype segregation. The segregation can include a dominant and additive segregation model and recessive segregation model. In one exemplary embodiment, finding at least one enriched pedigree based on dominant and additive segregation model comprises selecting pedigrees with one possible structure and at least three affecteds with a common ancestor. It can further comprise selecting at least one enriched pedigree with one or more related unaffecteds to reduce false positives. In another exemplary embodiment, finding at least one enriched pedigree based on recessive segregation model comprises selecting pedigrees with one possible structure and more than one affected with unaffected parents. It can further comprise selecting at least one enriched pedigree with at least two affected siblings to reduce false positives.

In some exemplary embodiments, the method for identifying a disease-causing variant comprises performing a segregation analysis to form a specific genetic model of segregation. The specific genetic model of segregation can include a dominant genetic model of segregation or a recessive genetic model of segregation. Additionally, specific genetic model of segregation could also include a genetic model of segregation based on other modes of inheritance, such as, P-linked, multifactorial or mitochondrial-linked mode of inheritance. In one exemplary embodiment, the method for identifying a disease-causing variant comprises performing a segregation analysis to form a dominant genetic model of segregation wherein the disease-causing variants segregate with the affecteds for at least one binary trait, an extreme quantitative trait, or a combination thereof. In one exemplary embodiment, the method for identifying a disease-causing variant comprises performing a segregation analysis to form a recessive genetic model of segregation wherein the disease-causing variants segregate with the affecteds who are biallelic variant carriers in given gene, and if genetic data is available for parents, they must be heterozygous for the identified disease-causing variant.

In some exemplary embodiments, the method for identifying a disease-causing variant can comprise performing segregation analysis to identify variant trait pairs that co-segregate within and across the at least one enriched pedigree. In one exemplary embodiment, the method for identifying a disease-causing variant comprises segregation analysis to identify variant trait pairs that co-segregate within and across multiple enriched pedigrees.

In some exemplary embodiments, the method for identifying a disease-causing variant can comprise performing segregation analysis to identify segregating variants or genes in other affecteds for the phenotype of interest not included in a family structure.

In some exemplary embodiments, the method for identifying a disease-causing variant can comprise performing segregation analysis which includes cross referencing variants and traits with association results from population-scale analyses.

In some exemplary embodiments, the method for identifying a disease-causing variant can comprise performing segregation analysis to identify previously known causal variants and genes.

In some exemplary embodiments, the method for identifying a disease-causing variant further can comprise prioritizing the enriched pedigrees by the number of supporting pedigrees/affecteds and by the number of candidate causal variants and genes.

In some exemplary embodiments, the method for identifying a disease-causing variant can comprise analyzing the variant trait pairs further comprises identifying sets of affecteds with sufficient family data to warrant a family-based association analysis.

In some exemplary embodiments, the method for identifying a disease-causing variant can comprise analyzing the variant trait pairs includes performing the Transmission Disequilibrium Test (TDT) or other analyses where appropriate based on pedigree and phenotype information.

In some exemplary embodiments, the method for identifying a disease-causing variant can include methods for identifying a disease-causing variant for several physiological disorders.

In one exemplary aspect, the disclosure provides a non-transitory computer readable medium storing instructions for causing a processor to perform a method for generating an enriched pedigree, comprises generating a first degree network of individuals based on exome sequencing data of a cohort, identifying individuals in the first degree network as an affected or an unaffected, and generating at least one enriched pedigree containing the individuals including designation as affected or unaffected.

In some exemplary embodiments, the non-transitory computer readable medium storing instructions for causing a processor to perform a method for generating an enriched pedigree comprises identifying whether or not individuals in the pedigree are affected or unaffected, wherein the individual with at least one binary trait is identified as affected and the individual without the at least one binary trait is identified as unaffected, and then evaluating whether the pattern of affected and unaffected individuals is consistent with a Mendelian mode of inheritance (e.g., autosomal dominant, autosomal recessive, x-linked dominant, x-linked recessive, or y-linked). In some specific exemplary embodiments, the binary trait can be defined using the International Statistical Classification of Diseases and Related Health Problems (ICD), a medical classification list by the World Health Organization (WHO) which contains codes for diseases, signs and symptoms, abnormal findings, complaints, social circumstances, and external causes of injury or diseases. The ninth or the tenth version of the ICD can be used to define the binary traits. In one exemplary embodiment, the individual for which no electronic health record data can be available for the specific binary trait or who has conflicting or unreliable data for the specific binary trait, irrespective of the absence or presence of the specific binary trait in the medical record, can be determined to be an unknown affected.

In some exemplary embodiments, the non-transitory computer readable medium storing instructions for causing a processor to perform a method for generating an enriched pedigree comprises identifying whether or not individuals in the pedigree are affected or unaffected, wherein the individual with at least one extreme quantitative trait is identified as affected and the individual without the at least one extreme quantitative trait is identified as unaffected, and then evaluating whether the pattern of affected and unaffected individuals is consistent with either a Mendelian mode of inheritance (e.g., autosomal dominant, autosomal recessive, x-linked dominant, x-linked recessive, or y-linked). Several parameters can be used to define whether or not someone is affected by an extreme quantitative trait, such as a maximum age cutoff to define an earlier onset of disorder, or having minimum or maximum or median measurement of the quantitative trait exceeded a defined statistical cutoff of deviation from normal population measurement of the trait (e.g., 2 standard deviations above the population mean). In one exemplary embodiment, the individual for which no electronic health record data can be available for the specific quantitative trait or who has conflicting or unreliable data for the specific quantitative trait, irrespective of the absence or presence of the specific quantitative trait in the medical record, can be determined to be an unknown affected.

In some exemplary embodiments, the non-transitory computer readable medium storing instructions for causing a processor to perform a method for generating an enriched pedigree comprises identifying whether or not individuals in the pedigree are affected or unaffected, wherein the individual with at least one binary trait, extreme quantitative trait or combination thereof is identified as affected and the individual without the at least one binary trait, extreme quantitative trait or combination thereof is identified as unaffected. The binary trait can be a defined ICD code as described above. Several parameters can be used to define extreme quantitative traits as described above. In one exemplary embodiment, the individual for whom no electronic health record data is available for the specific binary trait, quantitative trait, or combination thereof or who has conflicting or unreliable data for the specific binary trait, quantitative trait, or combination thereof, irrespective of the absence or presence of the specific quantitative trait in the medical record, can be determined to be an unknown affected.

In some exemplary embodiments, the non-transitory computer readable medium storing instructions for causing a processor to perform a method for generating an enriched pedigree comprises identifying whether or not individuals in the pedigree are affected or unaffected, wherein the individual with at least one binary trait, extreme quantitative trait or combination thereof is identified as affected and the individual without the at least one binary trait, extreme quantitative trait or combination thereof is identified as unaffected, and wherein the at least one binary trait, an extreme quantitative trait, or combination thereof can include two or more similar or complementary traits.

In some exemplary embodiments, the non-transitory computer readable medium storing instructions for causing a processor to perform a method for generating an enriched pedigree comprises identifying whether or not individuals in the pedigree are affected or unaffected, wherein the individual with at least one binary trait, extreme quantitative trait or combination thereof is identified as affected and the individual without the at least one binary trait, extreme quantitative trait or combination thereof is identified as unaffected, and wherein the at least one binary trait, an extreme quantitative trait, or combination thereof can include taking an intersection of two or more extreme or interesting traits.

In some exemplary embodiments, the non-transitory computer readable medium storing instructions for causing a processor to perform a method for generating an enriched pedigree can further comprise identifying an individual in the cohort to be affected if the individual has at least one binary trait, an extreme quantitative trait, or combination thereof and defining the individual determined to be affected as affected carrier of an association result from external analyses.

In some exemplary embodiments, the non-transitory computer readable medium storing instructions for causing a processor to perform a method for generating an enriched pedigree comprises generating a first degree network of individuals based on sequencing data of a cohort. The sequencing data can include whole genome sequencing data, exome sequencing data, or genotype data.

In some exemplary embodiments, the non-transitory computer readable medium storing instructions for causing a processor to perform a method for generating an enriched pedigree based on exome sequencing data. The first degree network of individuals based on exome sequencing data can be generated by leveraging the population's relatedness including: removing low-quality sequence variants from a dataset of nucleic acid sequence samples obtained from a plurality of human subjects, establishing an ancestral superclass designation for each of one or more of the samples, removing low-quality samples from the dataset, generating first identity-by-descent estimates of subjects within an ancestral superclass, generating second identity-by-descent estimates of subjects independent from subjects' ancestral superclass, and clustering subjects into primary first-degree family networks based on one or more of the second identity-by-descent estimates.

In some exemplary embodiments, the non-transitory computer readable medium storing instructions for causing a processor to perform a method for generating an enriched pedigree can comprise generating a first degree network of individuals based on sequencing data of a cohort wherein the cohort can include any dataset comprising a plurality of subjects.

In some exemplary embodiments, the non-transitory computer readable medium storing instructions for causing a processor to perform a method for generating an enriched pedigree can further include enriching the pedigree based on a p-value. The enrichment can include defining a “founder anchored branch” or “branch” of a pedigree as all descendants of a founder within a pedigree and using a binomial test to evaluate if the branch is enriched for a binary trait. The binary trait could be defined using the ICD as described above. The enrichment can also include defining a “founder anchored branch” or “branch” of a pedigree as all descendants of a founder within a pedigree and using a t-test to evaluate if the branch if enriched for an extreme quantitative trait. Several parameters can be used to define extreme quantitative traits as described above. Further, the enrichment can also include applying a multiple-test p-value cutoff.

In one exemplary aspect, the disclosure provides a non-transitory computer readable medium storing instructions for causing a processor to perform a method for identifying a disease-causing variant, comprises generating a first degree network of individuals based on exome sequencing data of a cohort, identifying individuals in the first degree network as an affected or an unaffected, creating at least one enriched pedigree containing the individuals including designation as affected or unaffected, performing segregation analysis to identify variant trait pairs that co-segregate within and across the at least one enriched pedigree, and analyzing the variant trait pairs to determine the disease-causing variant.

In some exemplary embodiments, the non-transitory computer readable medium storing instructions for causing a processor to perform a method for identifying a disease-causing variant comprises identifying whether or not individuals in the pedigree are affected or unaffected, wherein the individual with at least one binary trait is identified as affected and the individual without the at least one binary trait is identified as unaffected, and then evaluating whether the pattern of affected and unaffected individuals is consistent with a Mendelian mode of inheritance (e.g., autosomal dominant, autosomal recessive, x-linked dominant, x-linked recessive, or y-linked). In some specific exemplary embodiments, the binary trait can be defined using the International Statistical Classification of Diseases and Related Health Problems (ICD), a medical classification list by the World Health Organization (WHO) which contains codes for diseases, signs and symptoms, abnormal findings, complaints, social circumstances, and external causes of injury or diseases. The ninth or the tenth version of the ICD can be used to define the binary traits. In one exemplary embodiment, the individual for which no electronic health record data can be available for the specific binary trait or who has conflicting or unreliable data for the specific binary trait, irrespective of the absence or presence of the specific binary trait in the medical record, can be determined to be an unknown affected.

In some exemplary embodiments, the non-transitory computer readable medium storing instructions for causing a processor to perform a method for identifying a disease-causing variant comprises identifying whether or not individuals in the pedigree are affected or unaffected, wherein the individual with at least one extreme quantitative trait is identified as affected and the individual without the at least one extreme quantitative trait is identified as unaffected, and then evaluating whether the pattern of affected and unaffected individuals is consistent with either a Mendelian mode of inheritance (e.g., autosomal dominant, autosomal recessive, x-linked dominant, x-linked recessive, or y-linked). Several parameters can be used to define whether or not someone is affected by an extreme quantitative trait, such as a maximum age cutoff to define an earlier onset of disorder, or having minimum or maximum or median measurement of the quantitative trait exceeded a defined statistical cutoff of deviation from normal population measurement of the trait (e.g., 2 standard deviations above the population mean). In one exemplary embodiment, the individual for which no electronic health record data can be available for the specific quantitative trait or who has conflicting or unreliable data for the specific quantitative trait, irrespective of the absence or presence of the specific quantitative trait in the medical record, can be determined to be an unknown affected.

In some exemplary embodiments, the non-transitory computer readable medium storing instructions for causing a processor to perform a method for identifying a disease-causing variant comprises identifying whether or not individuals in the pedigree are affected or unaffected, wherein the individual with at least one binary trait, extreme quantitative trait or combination thereof is identified as affected and the individual without the at least one binary trait, extreme quantitative trait or combination thereof is identified as unaffected. The binary trait can be a defined ICD code as described above. Several parameters can be used to define extreme quantitative traits as described above. In one exemplary embodiment, the individual for whom no electronic health record data is available for the specific binary trait, quantitative trait, or combination thereof or who has conflicting or unreliable data for the specific binary trait, quantitative trait, or combination thereof, irrespective of the absence or presence of the specific quantitative trait in the medical record, can be determined to be an unknown affected.

In some exemplary embodiments, the non-transitory computer readable medium storing instructions for causing a processor to perform a method for identifying a disease-causing variant comprises identifying whether or not individuals in the pedigree are affected or unaffected, wherein the individual with at least one binary trait, extreme quantitative trait or combination thereof is identified as affected and the individual without the at least one binary trait, extreme quantitative trait or combination thereof is identified as unaffected, and wherein the at least one binary trait, an extreme quantitative trait, or combination thereof can include two or more similar or complementary traits.

In some exemplary embodiments, the non-transitory computer readable medium storing instructions for causing a processor to perform a method for identifying a disease-causing variant comprises identifying whether or not individuals in the pedigree are affected or unaffected, wherein the individual with at least one binary trait, extreme quantitative trait or combination thereof is identified as affected and the individual without the at least one binary trait, extreme quantitative trait or combination thereof is identified as unaffected, and wherein the at least one binary trait, an extreme quantitative trait, or combination thereof can include taking an intersection of two or more extreme or interesting traits.

In some exemplary embodiments, the non-transitory computer readable medium storing instructions for causing a processor to perform a method for identifying a disease-causing variant can further comprise identifying an individual in the cohort to be affected if the individual has at least one binary trait, an extreme quantitative trait, or combination thereof and defining the individual determined to be affected as affected carrier of an association result from external analyses.

In some exemplary embodiments, the non-transitory computer readable medium storing instructions for causing a processor to perform a method for identifying a disease-causing variant comprises generating a first degree network of individuals based on sequencing data of a cohort. The sequencing data can include whole genome sequencing data, exome sequencing data, or genotype data.

In some exemplary embodiments, the non-transitory computer readable medium storing instructions for causing a processor to perform a method for identifying a disease-causing variant based on exome sequencing data. The first degree network of individuals based on exome sequencing data can be generated by leveraging the population's relatedness including: removing low-quality sequence variants from a dataset of nucleic acid sequence samples obtained from a plurality of human subjects, establishing an ancestral superclass designation for each of one or more of the samples, removing low-quality samples from the dataset, generating first identity-by-descent estimates of subjects within an ancestral superclass, generating second identity-by-descent estimates of subjects independent from subjects' ancestral superclass, and clustering subjects into primary first-degree family networks based on one or more of the second identity-by-descent estimates.

In some exemplary embodiments, the non-transitory computer readable medium storing instructions for causing a processor to perform a method for identifying a disease-causing variant can comprise generating a first degree network of individuals based on sequencing data of a cohort wherein the cohort can include any dataset comprising a plurality of subjects.

In some exemplary embodiments, the non-transitory computer readable medium storing instructions for causing a processor to perform a method for identifying a disease-causing variant can further include enriching the pedigree based on a p-value. The enrichment can include defining a “founder anchored branch” or “branch” of a pedigree as all descendants of a founder within a pedigree and using a binomial test to evaluate if the branch is enriched for a binary trait. The binary trait could be defined using the ICD as described above. The enrichment can also include defining a “founder anchored branch” or “branch” of a pedigree as all descendants of a founder within a pedigree and using a t-test to evaluate if the branch if enriched for an extreme quantitative trait. Several parameters can be used to define extreme quantitative traits as described above. Further, the enrichment can also include applying a multiple-test p-value cutoff.

In some exemplary embodiments, the non-transitory computer readable medium storing instructions for causing a processor to perform a method for identifying a disease-causing variant can comprise identifying variant trait pairs that co-segregate with affecteds within the pedigree, and performing a segregation analysis which includes finding at least one enriched pedigree based on phenotype segregation. The segregation can include a dominant and additive segregation model and recessive segregation model. In one exemplary embodiment, finding at least one enriched pedigree based on dominant and additive segregation model comprises selecting pedigrees with one possible structure and at least three affecteds with a common ancestor. It can further comprise selecting at least one enriched pedigree with one or more related unaffecteds to reduce false positives. In another exemplary embodiment, finding at least one enriched pedigree based on recessive segregation model comprises selecting pedigrees with one possible structure and more than one affected with unaffected parents. It can further comprise selecting at least one enriched pedigree with at least two affected siblings to reduce false positives.

In some exemplary embodiments, the non-transitory computer readable medium storing instructions for causing a processor to perform a method for identifying a disease-causing variant can comprise performing a segregation analysis to form a specific genetic model of segregation. The specific genetic model of segregation can include a dominant genetic model of segregation or a recessive genetic model of segregation. Additionally, specific genetic model of segregation could also include a genetic model of segregation based on other modes of inheritance, such as, Y-linked, multifactorial or mitochondrial-linked mode of inheritance. In one exemplary embodiment, the method for identifying a disease-causing variant comprises performing a segregation analysis to form a dominant genetic model of segregation wherein the disease-causing variants segregate with the affecteds for at least one binary trait, an extreme quantitative trait, or a combination thereof. In one exemplary embodiment, the method for identifying a disease-causing variant comprises performing a segregation analysis to form a recessive genetic model of segregation wherein the disease-causing variants segregate with the affecteds who are biallelic variant carriers in given gene, and if genetic data is available for parents, they must be heterozygous for the identified disease-causing variant.

In some exemplary embodiments, the non-transitory computer readable medium storing instructions for causing a processor to perform a method for identifying a disease-causing variant can comprise performing a segregation analysis to identify variant trait pairs that co-segregate within and across the at least one enriched pedigree. In one exemplary embodiment, the method for identifying a disease-causing variant comprises segregation analysis to identify variant trait pairs that co-segregate within and across multiple enriched pedigrees.

In some exemplary embodiments, the non-transitory computer readable medium storing instructions for causing a processor to perform a method for identifying a disease-causing variant can comprise performing a segregation analysis to identify segregating variants or genes in other affecteds for the phenotype of interest not included in a family structure.

In some exemplary embodiments, the non-transitory computer readable medium storing instructions for causing a processor to perform a method for identifying a disease-causing variant can comprise performing a segregation analysis which includes cross referencing variants and traits with association results from population-scale analyses.

In some exemplary embodiments, the non-transitory computer readable medium storing instructions for causing a processor to perform a method for identifying a disease-causing variant can comprise performing a segregation analysis to identify previously known causal variants and genes.

In some exemplary embodiments, the non-transitory computer readable medium storing instructions for causing a processor to perform a method for identifying a disease-causing variant can comprise prioritizing the enriched pedigrees by the number of supporting pedigrees/affecteds and by the number of candidate causal variants and genes.

In some exemplary embodiments, the non-transitory computer readable medium storing instructions for causing a processor to perform a method for identifying a disease-causing variant can comprise analyzing the variant trait pairs further comprises identifying sets of affecteds with sufficient family data to warrant a family-based association analysis.

In some exemplary embodiments, the non-transitory computer readable medium storing instructions for causing a processor to perform a method for identifying a disease-causing variant can comprise analyzing the variant trait pairs includes performing the Transmission Disequilibrium Test (TDT) or other analyses where appropriate based on pedigree and phenotype information.

In some exemplary embodiments, the non-transitory computer readable medium storing instructions for causing a processor to perform a method for identifying a disease-causing variant for several physiological disorders.

In one exemplary aspect, the disclosure provides a system for generating an enriched pedigree, the system comprising a data processor and a memory coupled with the data processor, the processor being configured to generate a first degree network of individuals based on sequencing data of a cohort, identify whether individuals in the first degree network as an affected or an unaffected, and generate at least one enriched pedigree containing the individuals including designation as affected or unaffected.

In some exemplary embodiments, the system for generating an enriched pedigree comprises a data processor and a memory coupled with the data processor, the processor being configured to identify whether or not individuals in the pedigree are affected or unaffected, wherein the individual with at least one binary trait is identified as affected and the individual without the at least one binary trait is identified as unaffected, and then evaluating whether the pattern of affected and unaffected individuals is consistent with a Mendelian mode of inheritance (e.g., autosomal dominant, autosomal recessive, x-linked dominant, x-linked recessive, or y-linked). In some specific exemplary embodiments, the binary trait can be defined using the International Statistical Classification of Diseases and Related Health Problems (ICD), a medical classification list by the World Health Organization (WHO) which contains codes for diseases, signs and symptoms, abnormal findings, complaints, social circumstances, and external causes of injury or diseases. The ninth or the tenth version of the ICD can be used to define the binary traits. In one exemplary embodiment, the individual for which no electronic health record data can be available for the specific binary trait, or who has conflicting or unreliable data for the specific binary trait, irrespective of the absence or presence of the specific binary trait in the medical record, can be determined to be an unknown affected.

In some exemplary embodiments, the system for generating an enriched pedigree comprises a data processor and a memory coupled with the data processor, the processor being configured to identify whether or not individuals in the pedigree are affected or unaffected, wherein the individual with at least one extreme quantitative trait are identified as affecteds and the individual without the at least one extreme quantitative trait ereof are identified as unaffecteds, and then evaluating whether the pattern of affected and unaffected individuals is consistent with either a Mendelian mode of inheritance (e.g., autosomal dominant, autosomal recessive, x-linked dominant, x-linked recessive, or y-linked). Several parameters can be used to define whether or not someone is affected by an extreme quantitative trait, such as a maximum age cutoff to define an earlier onset of disorder, or having minimum or maximum or median measurement of the quantitative trait exceeded a defined statistical cutoff of deviation from normal population measurement of the trait (e.g., 2 standard deviations above the population mean). In one exemplary embodiment, the individual for which no electronic health record data can be available for the specific quantitative trait or who has conflicting or unreliable data for the specific quantitative trait, irrespective of the absence or presence of the specific quantitative trait in the medical record, can be determined to be an unknown affected.

In some exemplary embodiments, the system for generating an enriched pedigree comprises a data processor and a memory coupled with the data processor, the processor being configured to identify whether or not individuals in the pedigree are affected or unaffected, wherein the individual with at least one binary trait, extreme quantitative trait or combination thereof is identified as affected and the individual without the at least one binary trait, extreme quantitative trait or combination thereof is identified as unaffected. The binary trait can be a defined ICD code as described above. Several parameters can be used to define extreme quantitative traits as described above. In one exemplary embodiment, the individual for whom no electronic health record data is available for the specific binary trait, quantitative trait, or combination thereof or who has conflicting or unreliable data for the specific binary trait, quantitative trait, or combination thereof, irrespective of the absence or presence of the specific quantitative trait in the medical record, can be determined to be an unknown affected.

In some exemplary embodiments, the system for generating an enriched pedigree comprises a data processor and a memory coupled with the data processor, the processor being configured to identify individuals in the pedigree as affected or unaffected, wherein the individual with at least one binary trait, extreme quantitative trait or combination thereof is identified as affected and the individual without the at least one binary trait, extreme quantitative trait or combination thereof is identified as unaffected, and wherein the at least one binary trait, an extreme quantitative trait, or combination thereof can include two or more similar or complementary traits.

In some exemplary embodiments, the system for generating an enriched pedigree comprises a data processor and a memory coupled with the data processor, the processor being configured to identify individuals in the pedigree as affected or unaffected, wherein the individual with at least one binary trait, extreme quantitative trait or combination thereof is identified as affected and the individual without the at least one binary trait, extreme quantitative trait or combination thereof is identified as unaffected, and wherein the at least one binary trait, an extreme quantitative trait, or combination thereof can include taking an intersection of two or more extreme or interesting traits.

In some exemplary embodiments, the system for generating an enriched pedigree comprises a data processor and a memory coupled with the data processor, the processor being configured to identify an individual in the cohort to be affected if the individual has at least one binary trait, an extreme quantitative trait, or combination thereof and defining the individual determined to be affected as affected carrier of an association result from external analyses.

In some exemplary embodiments, the system for generating an enriched pedigree comprises a data processor and a memory coupled with the data processor, the processor being configured to generate a first degree network of individuals based on sequencing data of a cohort. The sequencing data can include whole genome sequencing data, exome sequencing data, or genotype data.

In some exemplary embodiments, the system for generating an enriched pedigree comprises a data processor and a memory coupled with the data processor, the processor being configured to generate a first degree network of individuals based on exome sequencing data. The first degree network of individuals based on exome sequencing data can be generated by leveraging the population's relatedness including: removing low-quality sequence variants from a dataset of nucleic acid sequence samples obtained from a plurality of human subjects, establishing an ancestral superclass designation for each of one or more of the samples, removing low-quality samples from the dataset, generating first identity-by-descent estimates of subjects within an ancestral superclass, generating second identity-by-descent estimates of subjects independent from subjects' ancestral superclass, and clustering subjects into primary first-degree family networks based on one or more of the second identity-by-descent estimates.

In some exemplary embodiments, the system for generating an enriched pedigree comprises a data processor and a memory coupled with the data processor, the processor being configured to generate a first degree network of individuals based on sequencing data of a cohort wherein the cohort can include any dataset comprising a plurality of subjects.

In some exemplary embodiments, the system for generating an enriched pedigree comprises a data processor and a memory coupled with the data processor, the processor being configured to further include enriching the pedigree based on a p-value. The enrichment can include defining a “founder anchored branch” or “branch” of a pedigree as all descendants of a founder within a pedigree and using a binomial test to evaluate if the branch is enriched for a binary trait. The binary trait could be defined using the ICD as described above. The enrichment can also include defining a “founder anchored branch” or “branch” of a pedigree as all descendants of a founder within a pedigree and using a t-test to evaluate if the branch if enriched for an extreme quantitative trait. Several parameters can be used to define extreme quantitative traits as described above. Further, the enrichment can also include applying a multiple-test p-value cutoff.

In one exemplary aspect, the disclosure provides a system for identifying disease causing variant comprises a data processor and a memory coupled with the data processor, the processor being configured to generate a first degree network of individuals based on sequencing data of a cohort, identify whether individuals in the first degree network as an affected or an unaffected, and generate at least one enriched pedigree containing the individuals including designation as affected or unaffected.

In some exemplary embodiments, the system for identifying a disease-causing variant comprises a data processor and a memory coupled with the data processor, the processor being configured to identify whether or not individuals in the pedigree are affected or unaffected, wherein the individual with at least one binary trait is identified as affected and the individual without the at least one binary trait is identified as unaffected, and then evaluating whether the pattern of affected and unaffected individuals is consistent with a Mendelian mode of inheritance (e.g., autosomal dominant, autosomal recessive, x-linked dominant, x-linked recessive, or y-linked). In some specific exemplary embodiments, the binary trait can be defined using the International Statistical Classification of Diseases and Related Health Problems (ICD), a medical classification list by the World Health Organization (WHO) which contains codes for diseases, signs and symptoms, abnormal findings, complaints, social circumstances, and external causes of injury or diseases. The ninth or the tenth version of the ICD can be used to define the binary traits. In one exemplary embodiment, the individual for which no electronic health record data can be available for the specific binary trait, or who has conflicting or unreliable data for the specific binary trait, irrespective of the absence or presence of the specific binary trait in the medical record, can be determined to be an unknown affected.

In some exemplary embodiments, the system for identifying a disease-causing variant comprises a data processor and a memory coupled with the data processor, the processor being configured to identify whether or not individuals in the pedigree are affected or unaffected, wherein the individual with at least one extreme quantitative trait are identified as affecteds and the individual without the at least one extreme quantitative trait ereof are identified as unaffecteds, and then evaluating whether the pattern of affected and unaffected individuals is consistent with either a Mendelian mode of inheritance (e.g., autosomal dominant, autosomal recessive, x-linked dominant, x-linked recessive, or y-linked). Several parameters can be used to define whether or not someone is affected by an extreme quantitative trait, such as a maximum age cutoff to define an earlier onset of disorder, or having minimum or maximum or median measurement of the quantitative trait exceeded a defined statistical cutoff of deviation from normal population measurement of the trait (e.g., 2 standard deviations above the population mean). In one exemplary embodiment, the individual for which no electronic health record data can be available for the specific quantitative trait or who has conflicting or unreliable data for the specific quantitative trait, irrespective of the absence or presence of the specific quantitative trait in the medical record, can be determined to be an unknown affected.

In some exemplary embodiments, the system for identifying a disease-causing variant comprises a data processor and a memory coupled with the data processor, the processor being configured to identify whether or not individuals in the pedigree are affected or unaffected, wherein the individual with at least one binary trait, extreme quantitative trait or combination thereof is identified as affected and the individual without the at least one binary trait, extreme quantitative trait or combination thereof is identified as unaffected. The binary trait can be a defined ICD code as described above. Several parameters can be used to define extreme quantitative traits as described above. In one exemplary embodiment, the individual for whom no electronic health record data is available for the specific binary trait, quantitative trait, or combination thereof or who has conflicting or unreliable data for the specific binary trait, quantitative trait, or combination thereof, irrespective of the absence or presence of the specific quantitative trait in the medical record, can be determined to be an unknown affected.

In some exemplary embodiments, the system for identifying a disease-causing variant comprises a data processor and a memory coupled with the data processor, the processor being configured to identify individuals in the pedigree as affected or unaffected, wherein the individual with at least one binary trait, extreme quantitative trait or combination thereof is identified as affected and the individual without the at least one binary trait, extreme quantitative trait or combination thereof is identified as unaffected, and wherein the at least one binary trait, an extreme quantitative trait, or combination thereof can include two or more similar or complementary traits.

In some exemplary embodiments, the system for identifying a disease-causing variant comprises a data processor and a memory coupled with the data processor, the processor being configured to identify individuals in the pedigree as affected or unaffected, wherein the individual with at least one binary trait, extreme quantitative trait or combination thereof is identified as affected and the individual without the at least one binary trait, extreme quantitative trait or combination thereof is identified as unaffected, and wherein the at least one binary trait, an extreme quantitative trait, or combination thereof can include taking an intersection of two or more extreme or interesting traits.

In some exemplary embodiments, the system for identifying a disease-causing variant comprises a data processor and a memory coupled with the data processor, the processor being configured to identify an individual in the cohort to be affected if the individual has at least one binary trait, an extreme quantitative trait, or combination thereof and defining the individual determined to be affected as affected carrier of an association result from external analyses.

In some exemplary embodiments, the system for identifying a disease-causing variant comprises a data processor and a memory coupled with the data processor, the processor being configured to generate a first degree network of individuals based on sequencing data of a cohort. The sequencing data can include whole genome sequencing data, exome sequencing data, or genotype data.

In some exemplary embodiments, the system for identifying a disease-causing variant comprises a data processor and a memory coupled with the data processor, the processor being configured to generate a first degree network of individuals based on exome sequencing data. The first degree network of individuals based on exome sequencing data can be generated by leveraging the population's relatedness including: removing low-quality sequence variants from a dataset of nucleic acid sequence samples obtained from a plurality of human subjects, establishing an ancestral superclass designation for each of one or more of the samples, removing low-quality samples from the dataset, generating first identity-by-descent estimates of subjects within an ancestral superclass, generating second identity-by-descent estimates of subjects independent from subjects' ancestral superclass, and clustering subjects into primary first-degree family networks based on one or more of the second identity-by-descent estimates.

In some exemplary embodiments, the system for identifying a disease-causing variant comprises a data processor and a memory coupled with the data processor, the processor being configured to generate a first degree network of individuals based on sequencing data of a cohort wherein the cohort can include any dataset comprising a plurality of subjects.

In some exemplary embodiments, the system for identifying a disease-causing variant comprises a data processor and a memory coupled with the data processor, the processor being configured to further include enriching the pedigree based on a p-value. The enrichment can include defining a “founder anchored branch” or “branch” of a pedigree as all descendants of a founder within a pedigree and using a binomial test to evaluate if the branch is enriched for a binary trait. The binary trait could be defined using the ICD as described above. The enrichment can also include defining a “founder anchored branch” or “branch” of a pedigree as all descendants of a founder within a pedigree and using a t-test to evaluate if the branch if enriched for an extreme quantitative trait. Several parameters can be used to define extreme quantitative traits as described above. Further, the enrichment can also include applying a multiple-test p-value cutoff.

In some exemplary embodiments, the system for identifying disease causing variant comprises a data processor and a memory coupled with the data processor, the processor being configured to identify variant trait pairs that co-segregate with affecteds within the pedigree, and performing a segregation analysis which includes finding at least one enriched pedigree based on phenotype segregation. The segregation can include a dominant and additive segregation model and recessive segregation model. In one exemplary embodiment, finding at least one enriched pedigree based on dominant and additive segregation model comprises selecting pedigrees with one possible structure and at least three affecteds with a common ancestor. It can further comprise selecting at least one enriched pedigree with one or more related unaffecteds to reduce false positives. In another exemplary embodiment, finding at least one enriched pedigree based on recessive segregation model comprises selecting pedigrees with one possible structure and more than one affected with unaffected parents. It can further comprise selecting at least one enriched pedigree with at least two affected siblings to reduce false positives.

In some exemplary embodiments, the system for identifying disease causing variant comprises a data processor and a memory coupled with the data processor, the processor being configured to perform a segregation analysis to form a specific genetic model of segregation. The specific genetic model of segregation can include a dominant genetic model of segregation or a recessive genetic model of segregation. Additionally, specific genetic model of segregation could also include a genetic model of segregation based on other modes of inheritance, such as, Y-linked, multifactorial or mitochondrial-linked mode of inheritance. In one exemplary embodiment, the method for identifying a disease-causing variant comprises performing a segregation analysis to form a dominant genetic model of segregation wherein the disease-causing variants segregate with the affecteds for at least one binary trait, an extreme quantitative trait, or a combination thereof. In one exemplary embodiment, the method for identifying a disease-causing variant comprises performing a segregation analysis to form a recessive genetic model of segregation wherein the disease-causing variants segregate with the affecteds who are biallelic variant carriers in given gene, and if genetic data is available for parents, they must be heterozygous for the identified disease-causing variant.

In some exemplary embodiments, the system for identifying disease causing variant comprises a data processor and a memory coupled with the data processor, the processor being configured to perform a segregation analysis to identify variant trait pairs that co-segregate within and across the at least one enriched pedigree. In one exemplary embodiment, the method for identifying a disease-causing variant comprises segregation analysis to identify variant trait pairs that co-segregate within and across multiple enriched pedigrees.

In some exemplary embodiments, the system for identifying disease causing variant comprises a data processor and a memory coupled with the data processor, the processor being configured to perform a segregation analysis to identify segregating variants or genes in other affecteds for the phenotype of interest not included in a family structure.

In some exemplary embodiments, the system for identifying disease causing variant comprises a data processor and a memory coupled with the data processor, the processor being configured to perform a segregation analysis which includes cross referencing variants and traits with association results from population-scale analyses.

In some exemplary embodiments, the system for identifying disease causing variant comprises a data processor and a memory coupled with the data processor, the processor being configured to perform a segregation analysis to identify previously known causal variants and genes.

In some exemplary embodiments, the system for identifying disease causing variant comprises a data processor and a memory coupled with the data processor, the processor being configured to prioritize the enriched pedigrees by the number of supporting pedigrees/affecteds and by the number of candidate causal variants and genes.

In some exemplary embodiments, the system for identifying disease causing variant comprises a data processor and a memory coupled with the data processor, the processor being configured to analyze the variant trait pairs further comprises identifying sets of affecteds with sufficient family data to warrant a family-based association analysis.

In some exemplary embodiments, the system for identifying disease causing variant comprises a data processor and a memory coupled with the data processor, the processor being configured to analyze the variant trait pairs includes performing the Transmission Disequilibrium Test (TDT) or other analyses where appropriate based on pedigree and phenotype information.

In some exemplary embodiments, the system for identifying disease causing variant comprises a data processor and a memory coupled with the data processor, the processor being configured to identify a diseases causing variants for several physiological disorders.

Methods and systems described herein can (i) provide a better understanding of molecular mechanisms causing disease, (ii) lead to better classification of disease and better management, (iii) provide identification of differential metabolism related to relevant gene variations (using critical enzymes or proteins or receptors associated with the altered metabolism in cancer cells as targets for new drug development), (iv) provide a refined class prediction for diseases like cancer which can help predict future clinical course and survival, and (v) design a gene therapy by identifying a genetic defect causing disease (by augmentation of desirable but deficient genes, or blocking of harmful genes (through anti-sense oligoribonucleotides or transcription factor decoys, or specific aptamers)).

### BRIEF DESCRIPTION OF THE DRAWINGS

The patent or application file contains at least one drawing executed in color. Copies of this patent or patent application publication with color drawing(s) will be provided by the Office upon request and payment of the necessary fee.

FIG. 1 is flow chart of an exemplary embodiment of the present invention to perform pedigree enrichment.

FIG. 2 is flow chart of an exemplary embodiment of the present invention to perform pedigree enrichment.

FIG. 3 is an exemplary operating environment.

FIG. 4 illustrates a plurality of system components configured for performing the disclosed methods.

FIG. 5 shows IBDO vs IDB1 plot for the first 92K sequenced individuals from the DiscovEHR cohort ascertained according to an exemplary embodiment.

FIG. 6 shows several enriched pedigrees from the DiscovEHR cohort for primary thrombophilia phenotype (Phe10_D685, ICD10CM D68.5) wherein pedigree enrichment is performed according to an exemplary embodiment.

FIGS. 7A and 7B show two enriched pedigrees for hereditary hemorrhagic telangiectasia phenotype (Phe10_I780, ICD10CM I78.0) wherein pedigree enrichment is performed according to an exemplary embodiment.

FIG. 8 shows a pedigree from the DiscovEHR cohort comprising the enriched pedigree demonstrating segregation of variant for hereditary hemorrhagic telangiectasia phenotype (Phe10_I780, ICD10CM I78.0) wherein pedigree enrichment and segregation analysis is performed according to an exemplary embodiment.

FIG. 9 shows several enriched pedigrees from the DiscovEHR cohort for emphysema phenotype wherein pedigree enrichment is performed according to an exemplary embodiment.

FIG. 10 shows an enriched pedigree from the DiscovEHR cohort for kidney transplant phenotype (Phe9_V420, ICD9CM V42.0) wherein pedigree enrichment is performed according to an exemplary embodiment.

FIG. 11 shows several enriched pedigrees from the DiscovEHR cohort for end stage renal disease phenotype (Phe9_5856, ICD9CM 585.6) wherein pedigree enrichment is performed according to an exemplary embodiment.

FIG. 12 shows an enriched pedigree from the DiscovEHR cohort for hereditary motor and sensory neuropathy phenotype (Charcot-Marie-Tooth Disease) (Phe10_G600, ICD10CM G60.0) phenotype.

FIG. 13 is a chart illustrating gene expression data of transcripts per million (TPM) of tropomyosin 2 (TMP2) gene encoded in various tissues

FIG. 14 shows an enriched pedigree from the DiscovEHR cohort for Bipolar Disorder wherein pedigree enrichment and segregation analysis are performed according to an exemplary embodiment.

FIG. 15 is a chart illustrating gene expression data of transcripts per million (TPM) of chromosome 20 open reading frame 203 (C20orf203) encoded in various tissues.

FIG. 16 shows an enriched pedigree from the DiscovEHR cohort for Bipolar Disorder phenotype wherein pedigree enrichment is performed according to an exemplary embodiment.

FIG. 17 shows an enriched pedigree from the DiscovEHR cohort for Bipolar Disorder phenotype wherein pedigree enrichment is performed according to an exemplary embodiment

FIG. 18 shows an enriched pedigree from the DiscovEHR cohort for Bipolar Disorder phenotype wherein pedigree enrichment is performed according to an exemplary embodiment

FIG. 19 is a chart illustrating gene expression data of transcripts per million (TPM) of microcephalin 1 (MCPH 1) in various tissues.

FIG. 20 shows an enriched pedigree from the DiscovEHR cohort for Familial thalassemia phenotype wherein pedigree enrichment is performed according to an exemplary embodiment.

FIG. 21 shows an enriched pedigree from the DiscovEHR cohort for Alkaline Phosphatase outpatient central tendency value wherein pedigree enrichment is performed according to an exemplary embodiment

### DETAILED DESCRIPTION

The term “a” should be understood to mean “at least one”; and the terms “about” and “approximately” should be understood to permit standard variation as would be understood by those of ordinary skill in the art; and where ranges are provided, endpoints are included.

Family-based association studies use a case-control design, with cases coming from a hospital or disease registry. Controls can be either unrelated (e.g., population or hospital/registry based) or are cases' family members (e.g., parents or siblings). The occurrence of a given allele in cases versus controls is compared to see if an “association” exists between genes and disease. With the availability of large-scale single-nucleotide polymorphisms (SNP) genotyping, association studies are increasingly common and are quickly expanding from focused candidate gene studies to genome-wide association studies.

The advent of next generation sequencing strategies has brightened up the prospects of elucidating the genetic defect in these diseases. A whole genome (approximately 3 billion base pairs) can currently be sequenced over a period of a few days and the costs are declining rapidly, making it accessible as a routine research tool. Sequencing the protein coding part of the genome, referred to as exome sequencing, is even more efficient for finding disease causing genes, because the exome represents only a small part of the genome (approximately 38 Mb) and because the exons harbor the vast majority of known mutations in Mendelian genes (Albert et al. Nature Methods (2007) 4:903-905; Gnirke et al. Nature Biotechnology (2009) 27: 182-189; Hodges et al. Nature Genetics (2007) 9: 1522-1527; Majewski et al. Journal of Medical Genetics (2011) 48: 580-589). Therefore, exome sequencing is highly suitable for the search for mutations in disorders with a suspected genetic cause without a priori knowledge of candidate genes or pathways being necessary.

Many of the large human sequencing studies collect samples from integrated health care populations that have accompanying phenotype-rich electronic health records (EHRs) with a goal of combining the EHRs and genomic sequence data to catalyze translational discoveries and precision medicine. The data from such projects can be used to identify certain genetic drivers of traits and diseases.

Spurious associations can be detected if cases and controls come from different source populations that have varying allele frequencies causing population stratification (Cardon and Palmer. Lancet (2003) 361(9357): 598-604). There is a debate regarding how much bias may result from such confounding (Wacholder et al. Cancer Epidemiology, Biomarkers & Prevention (2002) 11(6): 513-520; Thomas and Witte. Cancer Epidemiology, Biomarkers & Prevention (2002) 11(6): 502-512; Gorroochurn et al. Human Heredity (2004) 58(1): 40-48). Population stratification can be circumvented by using family-based study designs. When studying parents and their offspring or siblings, cases and controls within each family arise from the same source population. A common family-based case-control design is parent trios (e.g., the Transmission Disequilibrium Test (TDT) approach) and sibling controls. One could also study other relatives (e.g., cousins) or simultaneously study a large number of different family members.

Identifying families within a large cohort involves identifying pedigrees that consist of sufficient informative affected individuals for a given trait to be amenable for family-based genetic studies. Pedigrees are particularly informative when interrogating rare variants of potential moderate- to large-effect that co-segregate with a given phenotype of interest within a family. These pedigrees can be leveraged to help define subsets of related participants with phenotypes of interest and then examine these subsets to identify genetic drivers of traits and disease.

The disclosure is based, at least in part, on the recognition that information about first-degree network of individuals within a dataset of genomic samples of a plurality of subjects allows investigating the connection between rare genetic variations and diseases, among other things.

The methods described herein may be applied to various types of dataset of genomic samples. Non-limiting examples of types of dataset include single-healthcare-network-populations; multi-healthcare-network-populations; racially, culturally or socially homogeneous or heterogeneous populations; mixed-age populations or populations homogenous in terms of age; geographically concentrated or dispersed populations; or combination thereof. The dataset may have various types of genetic variant. Non-limiting examples of types of genetic variants that may be assessed include point mutations, insertions, deletions, inversions, duplications and multimerizations. Non-limiting examples of means by which the genetic variants may be acquired include the following steps:
Sample preparation and sequencing (Dewey et al. (2016), Science 354, aaf6814-1 to aaf6814-10);
Upon completion of sequencing, raw data from each sequencing run can be gathered in local buffer storage and uploaded to the DNAnexus platform (Reid et al. (2014); BMC Bioinformatics 15, 30) for automated analysis.
Sample-level read files can be generated with CASAVA (Illumina Inc., San Diego, Calif.) and aligned to GRCh38 with BWA-mem (Li and Durbin (2009); Bioinformatics 25, 1754-176; Li (2013); arXiv q-bio.GN).
The resultant BAM files can be processed using GATK (McKenna et al. (2010); Genome Res. 20, 1297-1303) and Picard to sort, mark duplicates, and perform local realignment of reads around putative indels.
Sequenced variants can be annotated with snpEFF (Cingolani et al. (2012); Fly (Austin) 6, 80-92) using Ensembl85 gene definitions to determine the functional impact on transcripts and genes.

The methods described herein may be applied for identifying a disease-causing variant responsible for a physiological disorder. Non-limiting examples include psychological disorders, blood-related disorders, pain-related disorders, hormone-related disorders, pulmonary diseases, dental disorders, fertility related disorders, mental disorders, movement disorders, cardiovascular disorders, circulatory disorders, autoimmune diseases, inflammatory diseases, renal disorders, hepatic disorders, hereditary hemorrhagic telangiectasia, motor sensory neuropathy, familial aortic aneurysms, thyroid cancer, pigmentary glaucoma, familial hypercholesterolemia, or combination thereof.

It is understood that the methods are not limited to any of the aforesaid steps, and that the acquisition of sequence variants may be conducted by any suitable means.

The disclosure is also based, at least in part, on the recognition that pedigrees generated from the information about first-degree relatives within a dataset of genomic samples of a plurality of subjects can provide information to identify rare variants segregating in families.

Several statistical methods have been developed that can be used to identify first degree relatives. One such non-limiting example is through calculation of Identity-by-decent (IBD) estimates if individuals to identify the different types of familial relationships within the dataset, and PRIMUS (Staples et al. (2014), Am. J. Hum. Genet. 95, 553-564) can be used to classify the pairwise relationships into different familial classes and to reconstruct the pedigrees. Only the estimated first-degree relationship among the dataset should be included. For example, to identify first-degree relatives from a dataset comprising exome sequencing data, the method as described in the co-pending U.S. Patent Publication No 20190205502 titled, “SYSTEMS AND METHODS FOR LEVERAGING RELATEDNESS IN GENOMIC DATA ANALYSIS” filed on Sep. 7, 2018, can be utilized, which is hereby incorporated by reference in its entirety.

In order to generate pedigrees form the dataset of genomic samples of a plurality of subjects, several approaches are available, such as, COP (Constructing Outbred Pedigrees) and CIP (Constructing Inbred Pedigrees), IPED (Inheritance Path-based Pedigree Reconstruction) and IPED2, PREPARE (Partitioning of Relatives), and Pedigree Reconstruction and Identification of the Maximally Unrelated Set (PRIMUS) (Riester et al. Bioinformatics (2009) 25: 2134-2139; Hadfield et al. Molecular Ecology (2006) 15: 3715-3730; Marshall et al. Molecular Ecology (1998) 7: 639-655; Cussens et al. Genetic Epidemiology (2013) 37: 69-83; He et al. Journal of Computational Biology (2013) 20: 780-792; Kirkpatrick et al. Journal of Computational Biology (2011) 18: 1481-1493; Staples et al. Genetic Epidemiology (2013) 37: 136-141; Shem-Tov and Halperin. PLoS Computational Biology (2014) 10: e1003610). Other methods, such as, PLINK, KING, and KINSHIP can also be used.

It is understood that this disclosure is not limited to any of the aforesaid dataset, methods of identifying first degree relatives and/or generating pedigrees, and that the acquisition and processing of dataset of genomic samples of a plurality of subject may be conducted by any suitable means known in the art.

The disclosure is also based, at least in part, on the recognition that information that generating pedigrees by determining the affecteds and unaffecteds in the dataset and refining the pedigrees to form enriched pedigrees is critical for down-stream analysis to find the connection between rare genetic variations and diseases, among other things.

The affecteds in the dataset can be defined by identifying the individuals in the dataset on the basis of the presence of at least one binary trait or an extreme quantitative trait or a combination thereof.

In some exemplary embodiments, the binary traits are defined using three letter codes from the International Statistical Classification of Diseases and Related Health Problems list (ICD). In some specific exemplary embodiments, three letter codes from 9^{th} or 10^{th} revision of the ICD were used to define the binary traits. The binary traits could further be defined using four letter codes from 9^{th} or 10^{th} revision of the ICD. An individual can be determined to be an “affected” if the individual's phenotype has the described binary trait. In some exemplary embodiments, the individual with the binary trait with a prevalence of over 5% in the cohort can be determined to be “unaffected” even if previously determined to be “affected”. Further, if the individual has indication of the absence or presence of the trait in the medical record and if the individual has conflicting records then the individual is determined to be an unknown affected.

In some exemplary embodiments, the extreme quantitative traits are defined by taking individuals with extremely high or low values of a trait based on the distribution of that trait in the population, e.g. calculating a z-score for each trait value and labeling individuals as “affected” if their traits' z-score is above 2 or below −2 for extremely high or low trait values, respectively. Further, if the individual has indication of the absence or presence of the trait in the medical record and if the individual has conflicting records then the individual is determined to be an unknown affected.

The pedigrees comprising the affecteds can further be refined to generate an enriched pedigree. The pedigree can be enriched based on phenotype segregation or p-value.

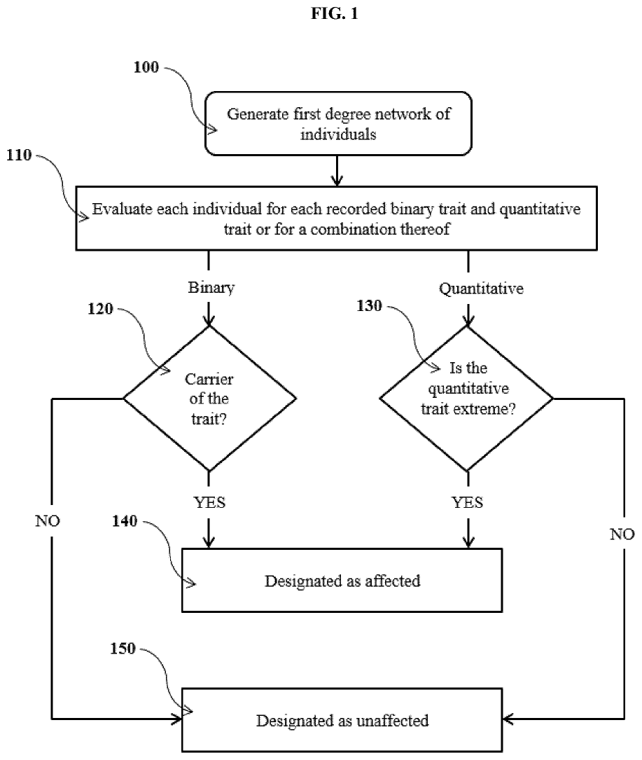

FIG. 1 is a flow chart of an exemplary embodiment wherein individuals from the first degree network are determined to be affecteds and unaffecteds. A first degree network of individuals is generated from a plurality of human subjects at step 100 by any suitable means. Every individual in the network can be evaluated for each recorded binary trait or each recorded quantitative trait or for a combination thereof at 110. Every individual in the network can be evaluated for each recorded binary trait at step 120 and is classified as “affected” if affected with the binary trait at step 140. On the contrary, if the individual is not affected with the specific binary trait under consideration, the individual is classified as “unaffected” at step 150. Every individual in the network can be evaluated for each recorded quantitative trait at step 130 and is classified as “affected” if affected with the quantitative trait at step 140. On the contrary, if the individual is not affected with the specific quantitative trait under consideration, the individual is classified as “unaffected” at step 150.

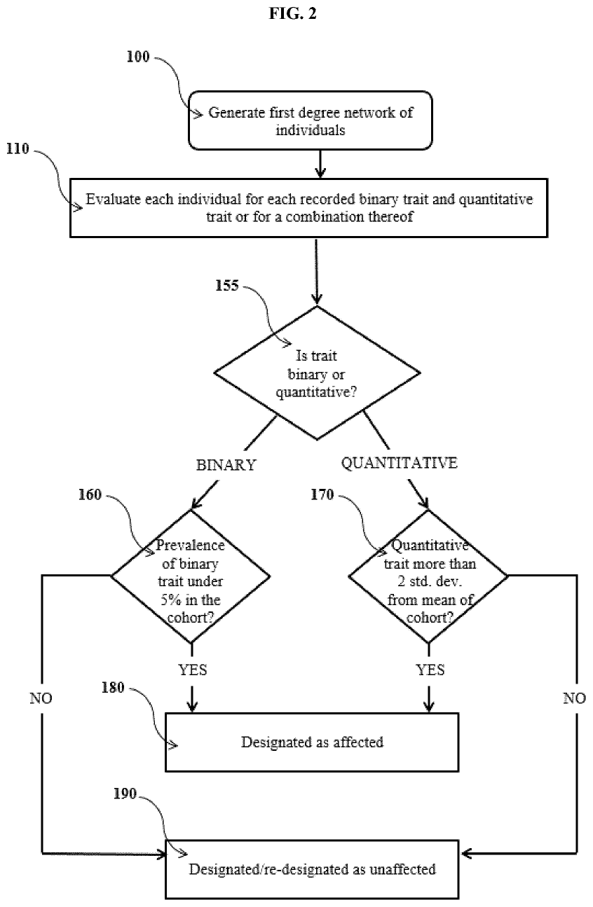

FIG. 2 is a flow chart of another exemplary embodiment wherein individuals from the first degree network are determined to be affecteds and unaffecteds. After generating a first degree network of individuals from a plurality of human subjects at step 100 by any suitable means, every individual in the network can be evaluated for each recorded binary trait or each recorded quantitative trait or for a combination thereof at 110. Further, every individual with any of the recorded binary trait or each recorded quantitative trait or for a combination thereof is evaluated on the basis of presence of the binary trait or quantitative trait at step 155. Following step 155, step 160 can classify the individual: if the binary trait used to classify the individual as affected has a prevalence of over 5% in the cohort, then the affected can be classified as “unaffected” at step 190; and if the binary trait used to classify the individual as affected has a prevalence of under 5%, then the affected can be classified as “affected” at step 180. Similarly, step 170 can reclassify the individual: if the quantitative trait used to classify the individual as affected is greater than two standard deviation than that a mean quantitative trait of the cohort then the individual is classified as “affected” at step 180 or else the individual is classified as “unaffected” at step 190.

Phenotype segregation within or across pedigrees can generate either a dominant and additive segregation model or a recessive segregation model. In some exemplary embodiments for pedigrees with phenotype segregation into a dominant and additive segregation model, the pedigrees with one possible structure and more than three affecteds with a common ancestor can be used to generate enriched pedigrees. Further, the enriched pedigrees can be prioritized for segregation analysis by selecting pedigrees with one or more than one related unaffected(s) to reduce false positives.

In some exemplary embodiments for pedigrees with phenotype segregation into a recessive segregation model, the pedigrees with one possible structure and more than one affecteds with unaffected parents are used to generate enriched pedigrees. Further, the enriched pedigrees can be prioritized for segregation analysis by selecting pedigrees with two or more than two affected siblings.

In some exemplary embodiments, the affecteds from two or more phenotypically similar or complementary binary or extreme quantitative traits can be merged to form affecteds for a disorder encompassing all those traits. For example, when looking for pedigrees enriched for Bipolar Disorder, unipolar disorder can also be considered since a genetic cause of Bipolar Disorder may only manifest as unipolar in some individuals.

In some exemplary embodiments, the affecteds with two or more extreme or interesting binary or extreme quantitative traits can be selected to form affecteds for a disorder encompassing all of those two or more traits. Taking the intersection of affecteds having two or more extreme or interesting traits may identify a more homogeneous subset of individuals. For example, to obtain an enriched pedigree with individuals with both asthma and COPD, the intersection of patients with both asthma and COPD are considered as affecteds.

It is understood that the disclosure is not limited to any of the aforesaid disorder or segregation model and that pedigree enrichment can conducted for any disorder or segregation model based on at least one binary trait, an extreme quantitative trait or a combination thereof.

Alternatively, enriched pedigrees can be determined based on p-value. In some exemplary embodiments, on identifying a founder anchored branch of the pedigree, a binomial test is carried out to evaluate if the pedigree is enriched for a binary trait. In other exemplary embodiments, on identifying a founder anchored branch of the pedigree, a t-test is carried out to evaluate if the pedigree is enriched for an extreme quantitative trait. Additionally, a multiple-test corrected p-value cutoff is set to remove false positives.

The disclosure is based, at least in part, on the recognition that a pedigree enriched for affected individuals with a given phenotype, an accompanying (e.g., rare) variant might segregate with and drive the phenotype of interest. Since such genetic cause may be more likely to be shared within a family unit, identification of pedigrees that are enriched for affecteds with phenotypes of interest can aid in identifying the casual (e.g., rare) mutation driving these phenotypes.

Once the enriched pedigrees have been identified, the underlying genetic cause can be determined by carrying out segregation analysis and family-based association analysis. For some pedigrees, there will be a known disease-causing mutation segregating with the affecteds. The remaining pedigrees can be prioritized by variants and genes that are segregating in affecteds across multiple pedigrees or with affects in the dataset that are not included in a pedigree. Regardless, the result from these segregation analyses can include a list of candidate variants.

Segregation analysis can be performed by testing models of varying degrees of generality. Models with various restrictions (e.g., dominant or recessive inheritance) can be compared to the most general model where all parameters in the model are estimated to see what model(s) best fit the data. Families with large pedigrees and many affected individuals are particularly informative both for establishing that genes are important and for identifying specific genes.

Methods that use pedigree structures to aid in identifying the genetic cause of a given phenotype typically involve innovative variations on association mapping, linkage analysis, or both. Such methods include MORGAN, pVAAST, FBAT (www.hsph.harvard.edu/fbat/fbat.htm), QTDT (csg.sph.umich.edu/abecasis/qtdt/), ROADTRIPS, rarelBD, and RV-GDT. The appropriate method to use depends on the phenotype, mode of inheritance, ancestral background, pedigree structure/size, number of pedigrees, and size of the unrelated dataset. In addition to using the relationships and pedigrees to directly interrogate gene-phenotype associations, they can also be used in a number of other ways to generate additional or improved data: pedigree-aware imputation, pedigree-aware phasing, Mendelian error checking, compound heterozygous knockout detection and de novo mutation calling, and variant calling validation.

Any of the methods described or exemplified by the present invention may be practiced as a computer-implemented method and/or as a system. Any suitable computer system known by the person having ordinary skill in the art may be used for this purpose.

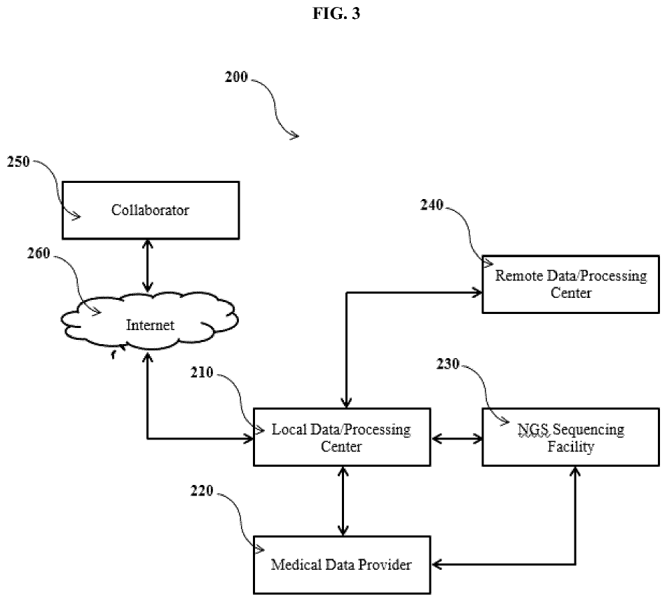

FIG. 3 illustrates various aspects of an exemplary environment 200 in which the present methods and systems can operate. The present methods may be used in various types of networks and systems that employ both digital and analog equipment. Provided herein is a functional description and that the respective functions can be performed by software, hardware, or a combination of software and hardware.

The environment 200 can comprise a Local Data/Processing Center 210. The Local Data/Processing Center 210 can comprise one or more networks, such as local area networks, to facilitate communication between one or more computing devices. The one or more computing devices can be used to store, process, analyze, output, and/or visualize biological data. The environment 200 can, optionally, comprise a Medical Data Provider 220. The Medical Data Provider 220 can comprise one or more sources of biological data. For example, the Medical Data Provider 220 can comprise one or more health systems with access to medical information for one or more patients. The medical information can comprise, for example, medical history, medical professional observations and remarks, laboratory reports, diagnoses, doctors' orders, prescriptions, vital signs, fluid balance, respiratory function, blood parameters, electrocardiograms, x-rays, CT scans, MRI data, laboratory test results, diagnoses, prognoses, evaluations, admission and discharge notes, and patient registration information. The Medical Data Provider 220 can comprise one or more networks, such as local area networks, to facilitate communication between one or more computing devices. The one or more computing devices can be used to store, process, analyze, output, and/or visualize medical information. The Medical Data Provider 220 can de-identify the medical information and provide the de-identified medical information to the Local Data/Processing Center 210. The de-identified medical information can comprise a unique identifier for each patient so as to distinguish medical information of one patient from another patient, while maintaining the medical information in a de-identified state. The de-identified medical information prevents a patient's identity from being connected with his or her particular medical information. The Local Data/Processing Center 210 can analyze the de-identified medical information to assign one or more phenotypes to each patient (for example, by assigning International Classification of Diseases “ICD” and/or Current Procedural Terminology “CPT” codes).

The environment 200 can comprise a NGS Sequencing Facility 230. The NGS Sequencing Facility 230 can comprise one or more sequencers (e.g., Illumina HiSeq 2500, Pacific Biosciences PacBio RS II, and the like). The one or more sequencers can be configured for exome sequencing, whole exome sequencing, RNA-seq, whole-genome sequencing, targeted sequencing, and the like. In an exemplary aspect, the Medical Data Provider 220 can provide biological samples from the patients associated with the de-identified medical information. The unique identifier can be used to maintain an association between a biological sample and the de-identified medical information that corresponds to the biological sample. The NGS Sequencing Facility 230 can sequence each patient's exome based on the biological sample. To store biological samples prior to sequencing, the NGS Sequencing Facility 230 can comprise a biobank (for example, from Liconic Instruments). Biological samples can be received in tubes (each tube associated with a patient), each tube can comprise a barcode (or other identifier) that can be scanned to automatically log the samples into the Local Data/Processing Center 210. The NGS Sequencing Facility 230 can comprise one or more robots for use in one or more phases of sequencing to ensure uniform data and effectively non-stop operation. The NGS Sequencing Facility 230 can thus sequence tens of thousands of exomes per year. In one aspect, the NGS Sequencing Facility 230 has the functional capacity to sequence at least 1000, 2000, 3000, 4000, 5000, 6000, 7000, 8000, 9000, 10,000, 11,000 or 12,000 whole exomes per month.

The biological data (e.g., raw sequencing data) generated by the NGS Sequencing Facility 230 can be transferred to the Local Data/Processing Center 210 which can then transfer the biological data to a Remote Data/Processing Center 240. The Remote Data/Processing Center 240 can comprise cloud-based data storage and processing center comprising one or more computing devices. The Local Data/Processing Center 210 and the NGS Sequencing Facility 230 can communicate data to and from the Remote Data/Processing Center 240 directly via one or more high capacity fiber lines, although other data communication systems are contemplated (e.g., the Internet). In an exemplary aspect, the Remote Data/Processing Center 240 can comprise a third party system, for example Amazon Web Services (DNAnexus). The Remote Data/Processing Center 240 can facilitate the automation of analysis steps, and allows sharing data with one or more Collaborators 250 in a secure manner. Upon receiving biological data from the Local Data/Processing Center 210, the Remote Data/Processing Center 240 can perform an automated series of pipeline steps for primary and secondary data analysis using bioinformatic tools, resulting in annotated variant files for each sample. Results from such data analysis (e.g., genotype) can be communicated back to the Local Data/Processing Center 210 and, for example, integrated into a Laboratory Information Management System (LIMS) can be configured to maintain the status of each biological sample.

The Local Data/Processing Center 210 can then utilize the biological data (e.g., genotype) obtained via the NGS Sequencing Facility 230 and the Remote Data/Processing Center 240 in combination with the de-identified medical information (including identified phenotypes) to identify associations between genotypes and phenotypes. For example, the Local Data/Processing Center 210 can apply a phenotype-first approach, where a phenotype is defined that may have therapeutic potential in a certain disease area, for example extremes of blood lipids for cardiovascular disease. Another example is the study of obese patients to identify individuals who appear to be protected from the typical range of comorbidities. Another approach is to start with a genotype and a hypothesis, for example that gene X is involved in causing, or protecting from, disease Y.

In an exemplary aspect, the one or more Collaborators 250 can access some or all of the biological data and/or the de-identified medical information via a network such as the Internet 260.

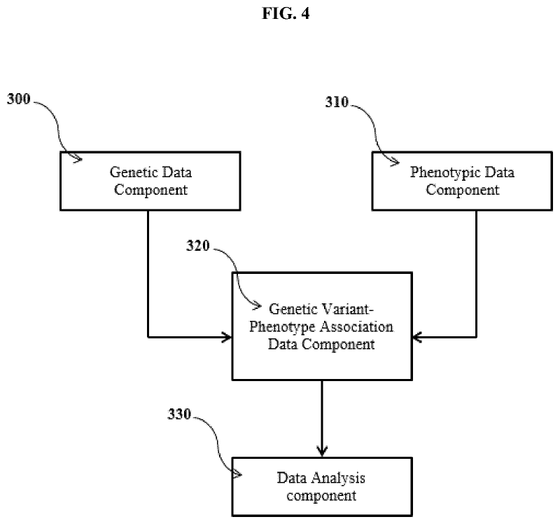

In an exemplary aspect, illustrated in FIG. 4, one or more of the Local Data/Processing Center 210 and/or the Remote Data/Processing Center 240 can comprise one or more computing devices that comprise one or more of a genetic data component 300, a phenotypic data component 310, a genetic variant-phenotype association data component 320, and/or a data analysis component 330. The genetic data component 300, the phenotypic data component 310, and/or the genetic variant-phenotype association data component 320 can be configured for one or more of, a quality assessment of sequence data, read alignment to a reference genome, variant identification, annotation of variants, phenotype identification, variant-phenotype association identification, data visualization, combinations thereof, and the like.

In an exemplary aspect, one or more of the components may take the form of an entirely hardware embodiment, an entirely software embodiment, or an embodiment combining software and hardware aspects. Furthermore, the methods and systems may take the form of a computer program product on a computer-readable storage medium having computer-readable program instructions (e.g., non-transitory computer software) embodied in the storage medium. More particularly, the present methods and systems may take the form of web-implemented computer software. Any suitable computer-readable storage medium may be utilized including hard disks, CD-ROMs, optical storage devices, or magnetic storage devices.

In an exemplary aspect, the genetic data component 300 can be configured for functionally annotating one or more genetic variants. The genetic data component 300 can also be configured for storing, analyzing, receiving, and the like, one or more genetic variants. The one or more genetic variants can be annotated from sequence data (e.g., raw sequence data) obtained from one or more patients (subjects). For example, the one or more genetic variants can be annotated from each of at least 100,000, 200,000, 300,000, 400,000 or 500,000 subjects. A result of functionally annotating one or more genetic variants is generation of genetic variant data. By way of example, the genetic variant data can comprise one or more Variant Call Format (VCF) files. A VCF file is a text file format for representing SNP, indel, and/or structural variation calls. Variants are assessed for their functional impact on transcripts/genes and potential loss-of-function (pLoF) candidates are identified. Variants are annotated with snpEff using the Ensembl75 gene definitions and the functional annotations are then further processed for each variant (and gene).

The consecutive labeling of method steps as provided herein with numbers and/or letters is not meant to limit the method or any embodiments thereof to the particular indicated order.

Various publications, including patents, patent applications, published patent applications, accession numbers, technical articles and scholarly articles are cited throughout the specification. Each of these cited references is incorporated by reference, in its entirety and for all purposes, herein.

The disclosure will be more fully understood by reference to the following Examples, which are provided to describe the disclosure in greater detail. They are intended to illustrate and should not be construed as limiting the scope of the disclosure.

### EXAMPLES

### Example 1

### Individuals and Samples

93,368 de-identified Geisinger Health System (GHS) participants who had given consent to be part of the MyCode Community Health Initiative were sequenced. As part of this initiative, individuals agreed to provide blood and DNA samples for broad, future research, including genomic analyses as part of the Regeneron GHS DiscovEHR collaboration and linking to data in the GHS EHR under a protocol approved by the Geisinger Institutional Review Board. All analyses performed were done in accordance with the participants' consent and IRB approval. Each participant has their exome linked to a corresponding de-identified EHR. The DiscovEHR study did not specifically target families as study participants but was implicitly enriched for adults who interact frequently with the healthcare system because or chronic health problems (and who might be related to each other) as well as participants from the Coronary Catheterization Laboratory and the Bariatric Service from GHS.

### Example 2

Sample Preparation, Sequencing, Variant calling, and Sample QC

Sample preparation and sequencing for the first 61Ksamples (“VCRome set”) have been previously described (Dewey et al. Science (2016) 354: aaf6814). The remaining set of 31K samples was prepared in the same process, except that in place of the NimbleGen probed capture, a slightly modified version of IDT's xGen probes were used with addition of supplemental probes to capture regions of the genome well covered by the NimbleGen VCRome capture reagent but poorly covered by the standard xGen probes. Captured fragments were bound to streptavidin-conjugated beads, and non-specific DNA fragment were removed by a series of stringent washes according to the manufacturer's (IDT's) recommended protocol. The second set of samples was referred to as the “xGen set.” Variant calls were produced with the Genome Analysis Toolkit (GATK; Web Resources). GATK was used for local realignment of the aligned, duplicate-marked reads of each sample around putative indels. INDEL realigned, duplicate-marked reads were processed using GATK's HaplotypeCaller to identify all exonic positions at which a sample varied from the genome reference in the genomic variant call format (gVCf). Genotyping was accomplished with GATK's GenotypeGYCFs on each sample and a training set of 50 randomly selected samples outputting a single-sample variant call format (VCF) file identifying both single-nucleotide variants (SNVs) and indels as compared to the reference. The single-sample VCF files were used to create a pseudo-sample that contained all variable sites from the single-sample VCF files in both sets. Independent pVCF files were created for the VCRome set by joint calling 200 single-sample gVCFfiles with the pseudo-sample to force a call or no-call for each sample at all variable sites across the two capture sets. All 200-sample pVCFfiles were combined to create the VCRome pVCF file and then repeated this process to create the xGen pVCF file. VCRome and xGen pVCF files were combined to create the union pVCF. Sequence reads to GRCh38 were aligned and annotated variants by using Ensembl 85 gene definitions. The gene definitions were restricted to 54,214 transcripts, corresponding to 19,467 genes that are protein-coding with an annotated start and stop. After the previously described sample QC process, 92,455 exomes remained for analysis.

### Example 3

### Principal Components and Ancestry Estimation

PLINKv1.910 was used to merge the union datasets with HapMap318 and, on the basis of reference SNP duster ID, SNPs that were in both datasets were kept. The analysis was restricted to high quality common SNPs with minor-allele frequency >10%, genotype missingness <5%, and a Hardy-Weinberg Equilibrium p value >0.00001 by applying the following PLINK filters: “-maf 0.1-geno 0.05-snps-only-h we 0.00001.” The principal components (PCs) for the HapMap3 samples were calculated and then projected each simple in the dataset on to those PCs by using PLINK. We used the PCs for the HapMap3 samples to train a kernel density estimator (KDE) for each of the five ancestral superclasses: African (AFR). admixed American (AMR), east Asian (EAS), European (EUR), and south Asian (SAS). The KDEs were calculated to estimate the likelihood that each sample belongs to each of the super classes. For each sample, ancestral superclass based on the basis of likelihoods was assigned. If a sample had two ancestral groups with a likelihood >0.3, then the sample was assigned AFR over EUR, AMR over EUR, AMR over EAS, SAS over EUR, and AMR over AFR; otherwise “UNKNOWN.” If zero or more than two ancestral groups had a high enough likelihood, then the sample was assigned “UNKNOWN” for ancestry. Samples with unknown ancestry were excluded from the ancestry based identity-by-descent (IBD) calculations.

### Example 4

### IBD Estimation

High-quality, common variants were filtered by running PLINK on the complete dataset using the following flags: -maf 0.1-geno 0.05-snps-only-hwe 0.00001. Then a two-pronged approach was taken to obtain accurate IBD estimates from the exome data. First, IBD estimates among individuals were calculated within the same ancestral superclass (e.g. AMR, AFR, EAS, EUR, and SAS) as determined from the ancestry analysis.

Second, in order to catch the first-degree relationships between individuals with different ancestries, IBD estimates were calculated among all individuals using the -min 0.3 PLINK option. Individuals were then grouped into first-degree family networks where network nodes were individuals and edges were first-degree relationships. Each first-degree family network was run through the prePRIMUS pipeline (Staples et al. (2014); Am. J. Hum. Genet. 95, 553-564), which matched the ancestries of the samples to appropriate ancestral minor allele frequencies to improve IBD estimation. This process accurately estimated first-degree relationships among individuals within each family network (minimum PI_HAT of 0.15).

### Example 5

### Relationship Estimation and Relatedness Description in a Cohort of 92K Human Exomes

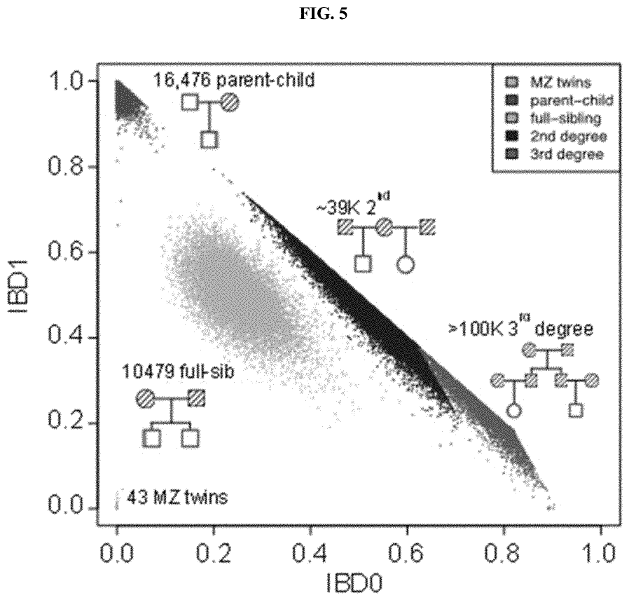

From the DiscovEHR dataset of 92,455 individuals, 43 monozygotic twins, 16,476 parent-child relationships, 10,479 full-sibling relationships, and 39,000 second-degree relationships were identified (FIG. 5). Individuals were treated as nodes and relationships as edges to generate undirected graphs. Using only first-degree relationships, 12,594 connected components were identified, which are referred to as first degree family networks. 39% of individuals in the DiscovEHR cohort had at least one first-degree relative in the dataset.

TABLE 1
(Ancestral breakdown of the DiscovEHR dataset)

| Ancestry class | # of samples | % of people |
|---|---|---|
| EUR | 88634 | 95.9% |
| AFR | 1984 | 2.1% |
| AMR | 959 | 1.0% |
| SAS | 196 | 0.2% |
| EAS | 194 | 0.2% |
| UNKNOWN | 488 | 0.5% |

TABLE 2
(complete breakdown of the ancestral backgrounds of individuals
involved in first-degree relationships in the DiscovEHR dataset)

| relationship | ancestries | count |
|---|---|---|
| MZ twins | EUR-EUR | 42 |
| MZ twins | SAS-SAS | 1 |
| Parent-child | EUR-EUR | 16028 |
| Parent-child | AFR-AFR | 115 |
| Parent-child | AFR-EUR | 86 |
| Parent-child | AMR-EUR | 83 |
| Parent-child | AMR-AMR | 43 |
| Parent-child | EUR-UNKNOWN | 43 |
| Parent-child | UNKNOWN-UNKNOWN | 20 |
| Parent-child | AFR-UNKNOWN | 13 |
| Parent-child | AMR-UNKNOWN | 13 |
| Parent-child | EAS-UNKNOWN | 13 |
| Parent-child | SAS-SAS | 11 |
| Parent-child | AFR-AMR | 5 |
| Parent-child | EUR-SAS | 2 |
| Parent-child | EAS-SAS | 1 |
| full-sibling | EUR-EUR | 10364 |
| full-sibling | AFR-AFR | 155 |
| full-sibling | AMR-EUR | 24 |
| full-sibling | AMR-AMR | 16 |
| full-sibling | UNKNOWN-UNKNOWN | 10 |
| full-sibling | AMR-UNKNOWN | 4 |
| full-sibling | SAS-SAS | 2 |
| full-sibling | EAS-EAS | 1 |
| full-sibling | EAS-UNKNOWN | 1 |
| full-sibling | EUR-SAS | 1 |
| full-sibling | EUR-UNKNOWN | 1 |

### Example 6

### Pedigree Reconstruction

All first-degree family networks identified within the DiscovEHR cohort were reconstructed with PRIMUSv1.9.0. The combined IBD estimates were provided to PRIMUS along within the genetically derived sex and EHR reported age. A relatedness cutoff of PI_HAT >0.375 was specified to limit the reconstruction to first-degree family networks.

Over 300 electronic health record (EHR) derived phenotypes segregating in a Mendelian fashion among these pedigrees were found from the dataset, providing over 2,000 potentially informative pedigree-phenotype pairings that enable traditional Mendelian analyses at a large scale.

### Example 7

### Pedigree Enrichment

Individuals from the first-degree family network were determined to be “affected” or “unaffected” for at least one binary trait, an extreme quantitative trait or a combination thereof. These sets of affecteds were intersected with the pedigrees to identify pedigrees enriched with enough affected individuals to be amenable to a family-based segregation analysis.

2,978 trait-pedigree enrichment pairs were recognized from the dataset (2,596 dominant and 382 recessive). Among these trait-pedigree enrichment pairs, there were 3,975 affected individuals with 1,015 different traits in 981 pedigrees. More than 50% of traits enriched in two or more pedigrees and 357 traits enriched in three or more pedigrees.

Additionally, among the 2,978 trait-pedigree enrichment pairs, 1,911 were binary trait-pedigree enrichment pairs with 809 different traits with 673 pedigrees. In the binary trait-pedigree enrichment pairs, the most enriched pedigree was for dental caries (N=46). Further among the 2,978 trait-pedigree enrichment pairs, 1,067 were quantitative trait-pedigree enrichment pairs with 206 different traits with 581 pedigrees. In the quantitative trait-pedigree enrichment pairs, the most enriched pedigree was for high triglyceride_Med_LabValue (N=19).

### 7.1 Primary Thrombophilia

Primary Thrombophilia is an inherited disorder of the haemostatic mechanism leading to thrombi formation (hypercoagulability state). This is commonly affects the venous system (e.g., deep vein thrombosis, pulmonary embolism).

Individuals in the population were determined to be affecteds based on the binary trait for primary thrombophilia (Phe10_D685, ICD10 4D).

From the pedigrees reconstructed (Table 3 and 4) using the method recited in Example 6, first-degree pedigrees were filtered to remove all pedigrees without only one possible structure and with less than three primary thrombophilia affecteds with a common ancestor to produce enriched pedigrees for primary thrombophilia. In the cohort, the prevalence for primary thrombophilia (Phe10_D685, ICD10CM D68.5) was 1.3%.

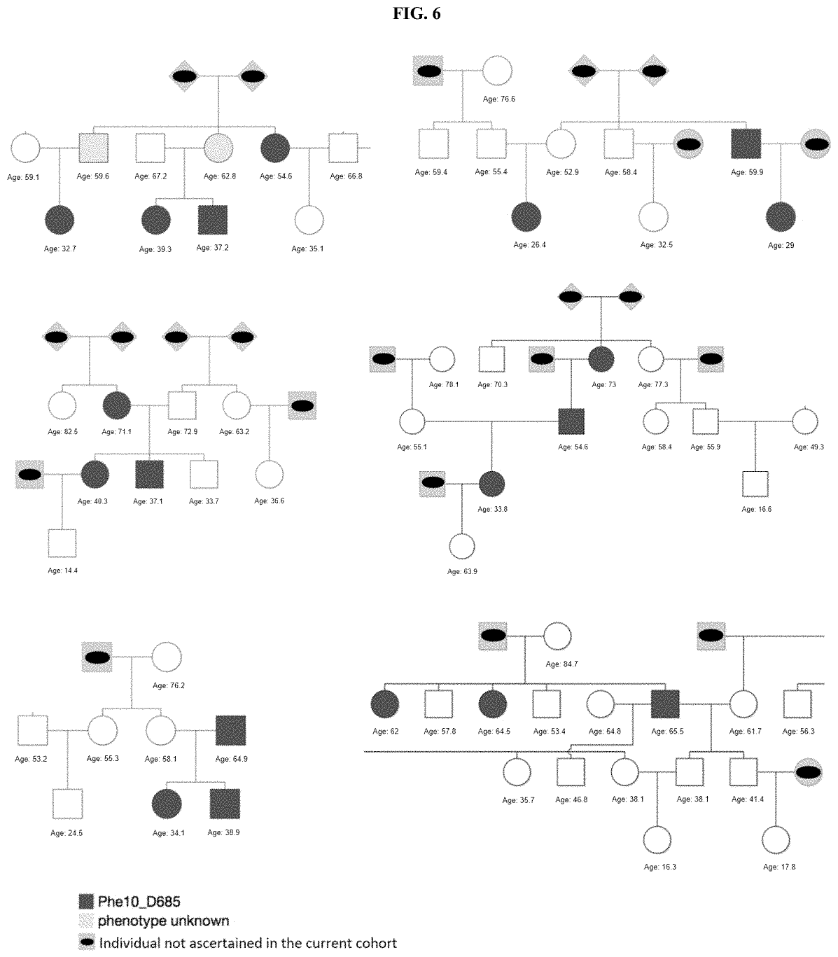

Several pedigrees enriched for primary thrombophilia were thus identified (See FIG. 6).

TABLE 3

| | | maximum | maximum | | |
|---|---|---|---|---|---|
| | | affecteds | informative | number of | |
| | | with | affecteds | affected | |
| Serial | number of | common | with common | sibling | pedigree |
| No. | affecteds | ancestor | ancestor | pairs | generations |
| 1 | 3 | 3 | 3 | 1 | 2 |
| 2 | 3 | 3 | 4 | 2 | 3 |
| 3 | 3 | 3 | 3 | 0 | 3 |
| 4 | 2 | 2 | 2 | 1 | 3 |
| 5 | 4 | 4 | 4 | 0 | 3 |
| 6 | 3 | 3 | 3 | 3 | 4 |
| 7 | 3 | 3 | 3 | 0 | 4 |
| 8 | 3 | 3 | 3 | 1 | 3 |

TABLE 4

| | | | | | number of | |
|---|---|---|---|---|---|---|
| | | | number of | number of | age consistent | |
| | affected | number of | unknown | samples in | possible | mode of |
| Serial No. | generations | unaffecteds | affecteds | pedigree | pedigrees | inheritance |
| 1 | 2 | 3 | 0 | 6 | 1 | dominant |
| 2 | 2 | 11 | 0 | 14 | 1 | dominant |
| 3 | 2 | 6 | 0 | 9 | 1 | dominant |
| 4 | 1 | 5 | 1 | 8 | 1 | recessive |
| 5 | 3 | 0 | 0 | 4 | 1 | dominant |
| 6 | 1 | 29 | 2 | 34 | 1 | dominant |
| 7 | 3 | 23 | 1 | 27 | 0 | dominant |
| 8 | 2 | 6 | 0 | 9 | 1 | dominant |

### 7.2 Hereditary Hemorrhagic Telangiectasia

Hereditary hemorrhagic telangiectasia (HTT) is a rare autosomal dominant disorder that affects blood vessels throughout the body (causing vascular dysplasia) and results in a tendency for bleeding. (The condition is also known as or Osler-Weber-Rendu disease (OWRD); the two terms are used interchangeably.) HHT is manifested by mucocutaneous telangiectases and arteriovenous malformations (AVMs), a potential source of serious morbidity and mortality. Lesions can affect the nasopharynx, central nervous system (CNS), lung, liver, and spleen, as well as the urinary tract, gastrointestinal (GI) tract, conjunctiva, trunk, arms, and fingers.

Individuals in the population were determined to be affecteds based on the binary trait for HTT (Phe10_I780, ICD10CM I78.0).

Two pedigrees were reconstructed (See Table 5 and 6) using the method recited in example 6 for HTT. Both the pedigrees had three HHT affecteds with a common ancestor and one possible structure. Further, in the cohort, the prevalence for HTT was 0.0%.

TABLE 5

| | | maximum | maximum | | |
|---|---|---|---|---|---|
| | | affecteds | informative | number of | |
| | | with | affecteds | affected | |
| Serial | number of | common | with common | sibling | pedigree |
| No. | affecteds | ancestor | ancestor | pairs | generations |
| 1 | 3 | 3 | 3 | 0 | 3 |
| 2 | 3 | 3 | 3 | 0 | 3 |

TABLE 6

| | | | | | number of | |
|---|---|---|---|---|---|---|
| | | | number of | number of | age consistent | |
| | affected | number of | unknown | samples in | possible | mode of |
| Serial No. | generations | unaffecteds | affecteds | pedigree | pedigrees | inheritance |
| 1 | 3 | 1 | 1 | 5 | 1 | dominant |
| 2 | 2 | 13 | 1 | 17 | 1 | dominant |

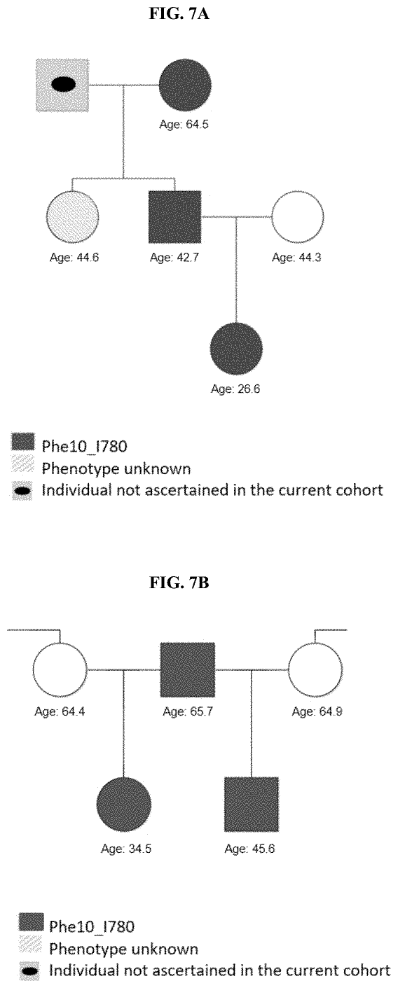

The two pedigrees enriched for binary trait for HTT were used to perform a rare variant segregation analysis (See FIGS. 7A and 7B).

For the pedigree enriched for HTT displayed in FIG. 7A, the segregation and association analysis indicated that the variant in SMAD4 gene co-segregated with the HTT phenotype in the pedigree (See Table 7). SMAD4 (SMAD family member 4) is a member of the SMAD family of signal transduction proteins. Smad proteins are phosphorylated and activated by transmembrane serine-threonine receptor kinases in response to transforming growth factor (TGF)-beta signaling. SMAD4 forms homomeric complexes and heteromeric complexes with other activated Smad proteins, which then accumulate in the nucleus and regulate the transcription of target genes and is an important component of the BMP signaling pathway. Mutations or deletions in SMAD4 have been associated with the genetic disorders hereditary hemorrhagic telangiectasia syndrome (HHT) and Myhre syndrome; and familial cancer susceptibility disorders including juvenile polyposis syndrome (heterozygous mutation in the SMAD4 gene on chromosome 18q21). SMAD4 acts as a tumor suppressor and inhibits epithelial cell proliferation. It may also have an inhibitory effect on tumors by reducing angiogenesis and increasing blood vessel hyper permeability. Somatic mutations in SMAD4 have been identified in pancreatic cancer.

TABLE 7

| GENE | GENE_NAME | NT_CHANGE | AA_CHANGE | TGP_FREQ | EXAC_FREQ |
|---|---|---|---|---|---|
| SMAD4 | SMAD | c.1242_1245delAGAC | p.D415Efs*20 | na | na |
| | family | | | | |
| | member 4 | | | | |

| GENE | RGC_FREQ | CONSERV | PREDICTION | GENOTYPES |
|---|---|---|---|---|
| SMAD4 | 0.000011 | na | deleterious | HET = 4/HOM = 0 |

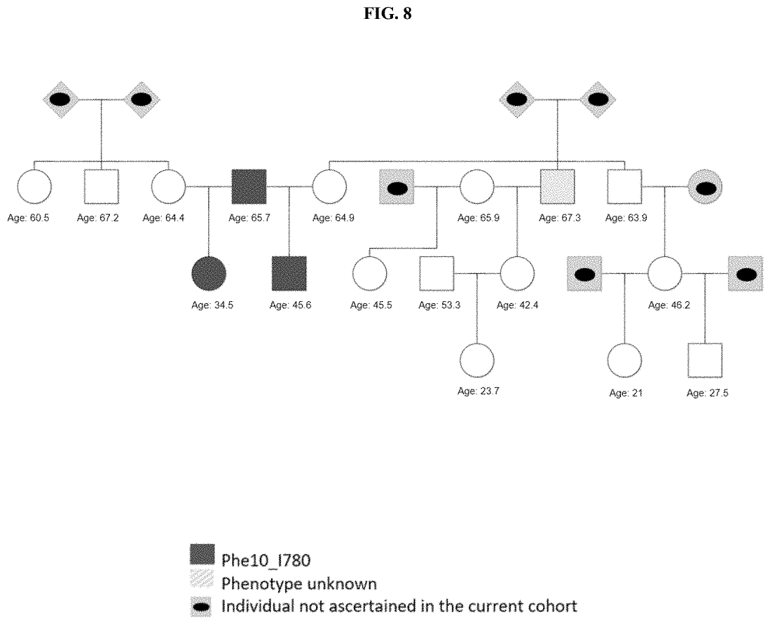

For the pedigree enriched for HTT displayed in FIG. 7B and FIG. 8 the segregation and association analysis indicated that the variant for activin A receptor type II-like 1 (ACVRL1) gene co-segregated with the HTT phenotype in the pedigree (See Table 8). ACVRL1 gene encodes a type I cell-surface receptor for the TGF-beta superfamily of ligands and shares similar domain structures with other closely related ALK or activin receptor-like kinase proteins that form a subfamily of receptor serine/threonine kinases. Mutations in ACVRL1 are associated with hemorrhagic telangiectasia type 2, also known as Rendu-Osler-Weber syndrome 2 and pulmonary arterial hypertension. Patients present with conjunctival telangiectasia, nasal mucosa telangiectases often leading to nose bleeding as the first sign of disease, mouth telangiectases, arteriovenous malformations in a variety of organs, skin telangiectases, anemia, and some develop pulmonary arterial hypertension. Visceral findings of HHT2 included pulmonary arteriovenous malformations (PAVMs), cerebral AVM, spinal AVM, hepatic AVM, gastrointestinal bleeding due to AVMs, and cirrhosis. Neurological manifestations of HHT2 include seizures, ischemic stroke, migraine,—cerebral arteriovenous malformation, and intracerebral hemorrhages.

TABLE 8

| GENE | GENE_NAME | NT_CHANGE | AA_CHANGE | TGP_FREQ | EXAC_FREQ |
|---|---|---|---|---|---|
| ACVRL1 | activin A | c.C853T | p.L285F | na | na |
| | receptor | | | | |
| | like type 1 | | | | |

| GENE | RGC_FREQ | CONSERV | PREDICTION | GENOTYPES |
|---|---|---|---|---|
| ACVRL1 | 0.000011 | conserved | deleterious | HET = 4/HOM = 0 |

7.3 Emphysema in Patients with GOLD Stage 2-4 by Spirometry

Emphysema is a lung condition that causes shortness of breath and one of the diseases that comprises chronic obstructive pulmonary disease (COPD). In people with emphysema, the air sacs in the lungs (alveoli) are damaged. Over time, the inner walls of the air sacs weaken and rupture—creating larger air spaces instead of many small ones. This reduces the surface area of the lungs and, in turn, the amount of oxygen that reaches your bloodstream. On exhalation, the damaged alveoli don't work properly and old air becomes trapped, leaving no room for fresh, oxygen-rich air to enter.

Binary traits for “Emphysema in Patients with GOLD Stage 2-4 by Spirometry” were derived from the quantitative traits for pulmonary function test. A high confidence set of non-smoking COPD patients based on multiple incidences reported in their electronic medical records was used. One of the quantitative traits for pulmonary function test was defined using “Pre-Bronchodilator Forced Expiratory Flow at 50 percent Forced Vital Capacity to Forced Inspiratory Flow at 50 percent Forced Vital Capacity from most recent spirometry.” The mean for the trait in the population was 0 and the standard deviation was 0.27. The enrichment was performed using the lower limit of the quantitative trait. Another quantitative trait for pulmonary function test was defined using “Percent of Predicted Post-Bronchodilator Forced Expiratory Volume in 1 second from most recent spirometry.” The mean for the trait in the population was 81.89 and the standard deviation was 20.84. The enrichment was performed using the lower limit of the quantitative trait.

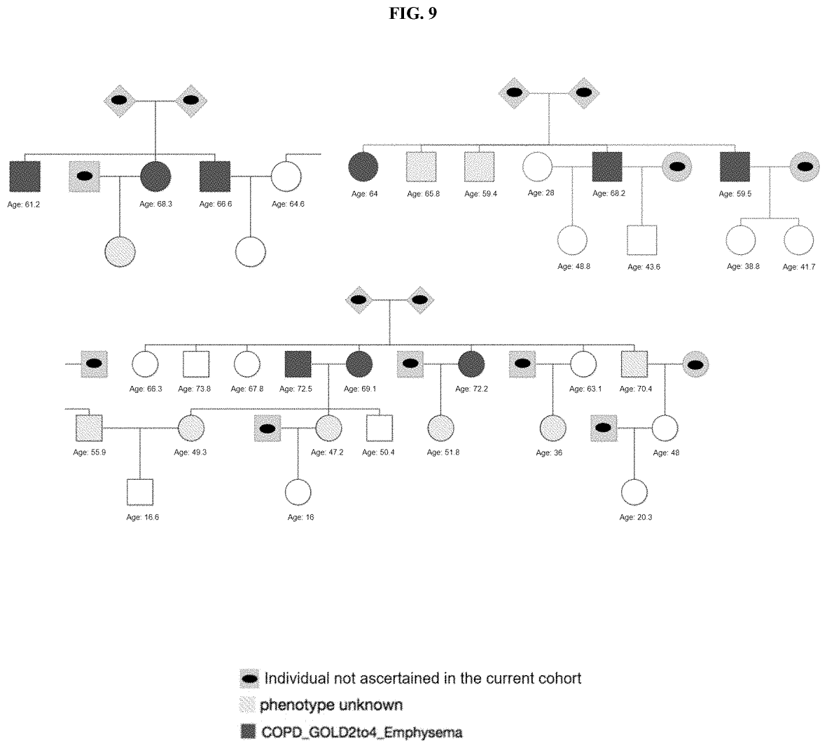

The pedigrees enriched for binary trait for Emphysema in Patients with GOLD Stage 2-4 by Spirometry from the first degree family network were isolated (See FIG. 9). In the cohort, the prevalence for this particular phenotype was 1.8%. The pedigrees had only one possible structure and comprised three affecteds with a common ancestor.

### 7.4 Kidney Transplant

A pedigree enriched for binary trait for kidney transplant (Phe9_V420, ICD9DM V42.0) was isolated from the first degree family network. The prevalence for this particular phenotype was 0.8%.

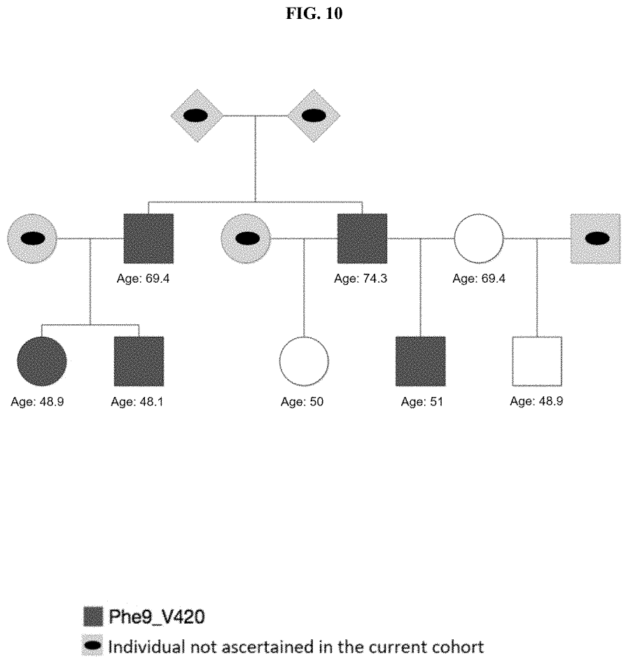

The first-degree pedigree had only one possible structure and had four affecteds with a common ancestor. The pedigree comprising the required criteria was identified (See FIG. 10 and Table 9).

TABLE 9

| | Factors influencing health |
|---|---|
| | status and contact with |
| Trait category | health services |
| number of affecteds | 5 |
| maximum affecteds with common ancestor | 5 |
| maximum informative affecteds with | 5 |
| common ancestor | |
| number of affected sibling pairs | 2 |
| pedigree generations | 2 |
| affected generations | 2 |
| number of unaffecteds | 3 |
| number of unknown affecteds | 0 |
| number of samples in pedigree | 8 |
| number of age consistent possible pedigrees | 1 |
| mode of inheritance | dominant |

### 7.5 End Stage Renal Disease

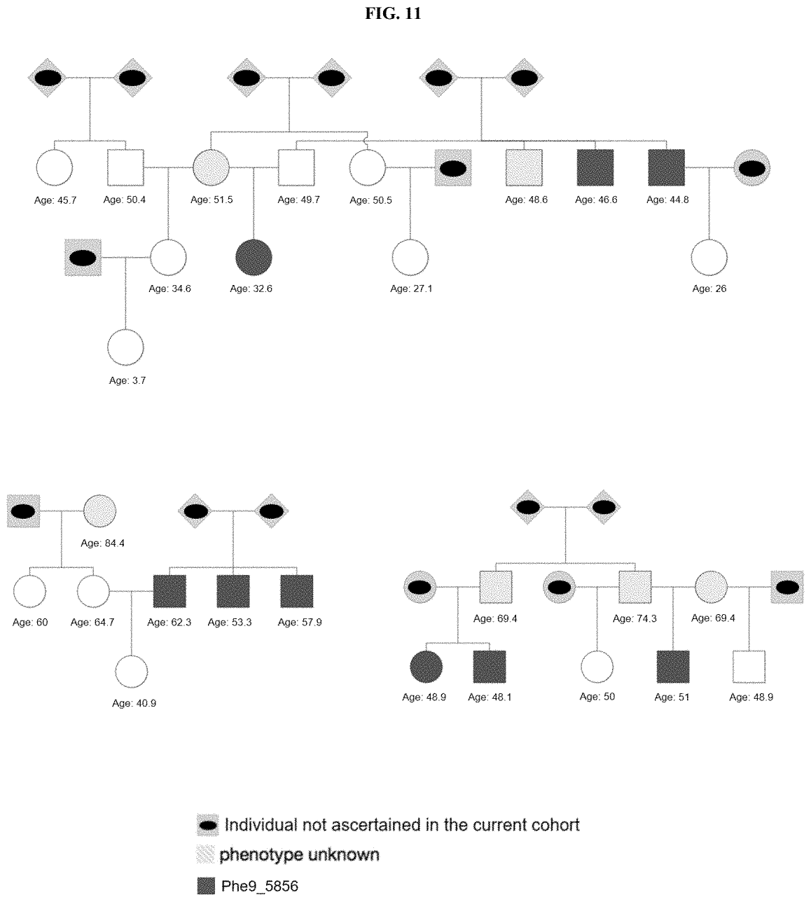

Individuals in the population were determined to be affecteds based on the binary trait for end stage renal disease (Phe10_5856, ICD9CM 585.6). Several pedigrees enriched for end stage renal disease were identified (FIG. 11).

### 7.6 Hereditary Motor and Sensory Neuropathy (Charcot-Marie-Tooth Disease)

Charcot-Marie-Tooth disease (CMT) is one of the most common inherited neurological disorders, affecting approximately 1 in 2,500 people in the United States. It is also known as hereditary motor and sensory neuropathy (HMSN) or peroneal muscular atrophy, comprises a group of disorders that affect peripheral nerves.

Individuals in the population were determined to be affecteds based on the binary trait for hereditary motor and sensory neuropathy (Phe10_G600, ICD10CM G60.0). In the cohort, the prevalence for this particular phenotype was 0.1%.

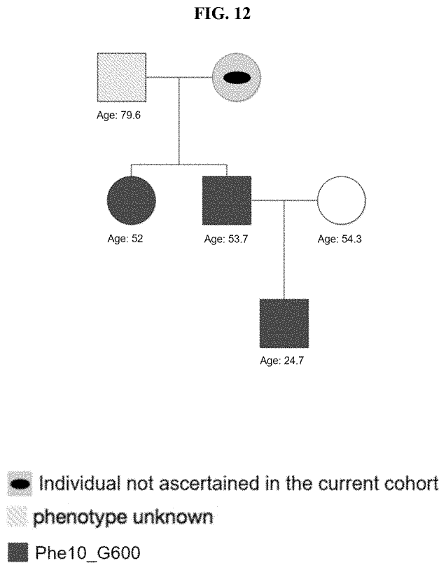

From the pedigrees reconstructed from example 6, the first-degree pedigree for hereditary motor and sensory neuropathy had one possible structure and three affecteds with a common ancestor (See FIG. 12 and Table 10).

TABLE 10

| | Diseases Of The |
|---|---|
| | Nervous System |
| Trait Category | And Sense Organs |
| number of affecteds | 3 |
| maximum affecteds with common ancestor | 3 |
| maximum informative affecteds with common | 3 |
| ancestor | |
| number of affected sibling pairs | 1 |
| pedigree generations | 3 |
| affected generations | 2 |
| number of unaffecteds | 1 |
| number of unknown affecteds | 1 |
| number of samples in pedigree | 5 |
| number of age consistent possible pedigrees | 1 |
| mode of inheritance | dominant |

For pedigree enriched for hereditary motor and sensory neuropathy, the segregation and association analysis indicated that the variant for tropomyosin 2 (beta) (TPM2) gene co-segregated with the hereditary motor and sensory neuropathy phenotype in the pedigree (Table 11). TPM2 encodes beta-tropomyosin, a member of the actin filament binding protein family, and mainly expressed in slow, type 1 muscle fibers. Mutations in TPM2 can alter the expression of other sarcomeric tropomyosin proteins, and cause cap disease, nemaline myopathy and distal arthrogryposis syndromes.

TABLE 11

| GENE | GENE_NAME | NT_CHANGE | AA_CHANGE | TGP_FREQ | EXAC_FREQ |
|---|---|---|---|---|---|
| TPM2 | tropomyosin 2 (beta) | c.G479A | p.R160H | na | 0.00002 |

| GENE | RGC_FREQ | CONSERV | PREDICTION | GENOTYPES |
|---|---|---|---|---|
| TPM2 | 0.00001 | conserved | deleterious | HET = 6/HOM = 0 |

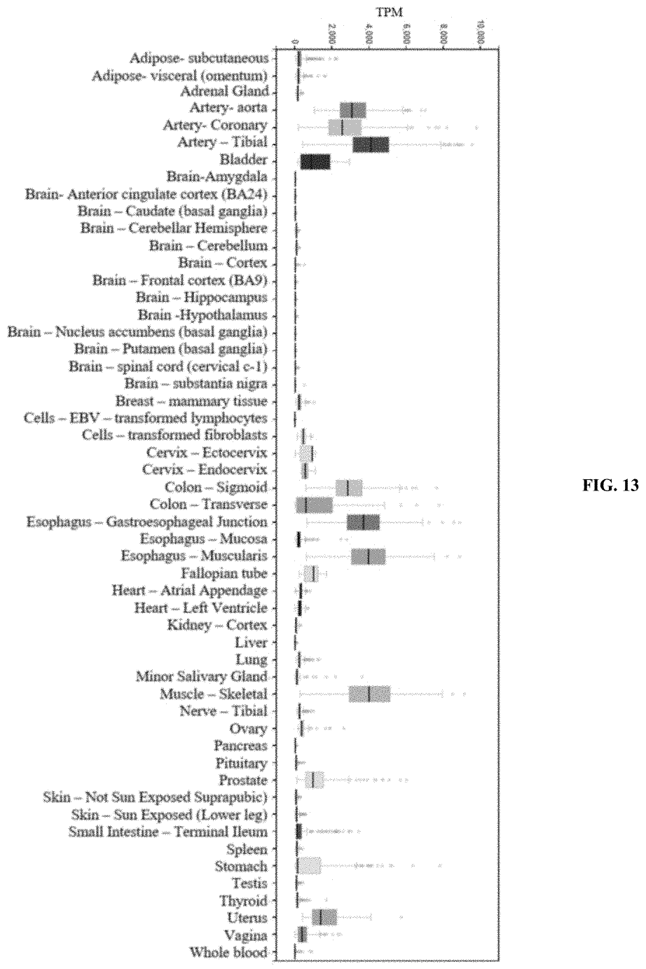

The gene expression data of transcripts per million (TPM) of TPM2 encoded in various tissues indicated a high occurrence in arteries, colon-sigmoid, esophagus-gastrointestinal junction, esophagus-muscularis, and skeletal muscle (See FIG. 13).

Patient records for the affecteds in the pedigree (See Table 12), suggested that this family does not show evidence of hereditary motor and sensory neuropathy, but rather they have Nemaline myopathy type 4 due to mutation in TPM2 (Donner et al. Neuromuscular Disorders (2009) 19: 348-3351).

TABLE 12

| Serial | |
|---|---|
| No. | Phenotype Notes |
| 1 | Tarsal tunnel syndrome; Hereditary peripheral neuropathy; |
| | Congenital hereditary muscular dystrophy; Acquired foot |
| | deformity; Congenital hereditary muscular dystrophy; |
| | Contracture of joint, multiple sites; Muscle weakness |
| | (generalized); Pain in limb; Peroneal muscular atrophy; |
| | Shortness of breath |
| 2 | Peroneal muscular atrophy; Cavovarus deformity of foot, acquired; |
| | Hereditary progressive muscular dystrophy; Mononeuritis of |
| | unspecified site; Pain in joint, ankle and foot; Pain in limb; |
| | Polyneuropathy in other diseases classified elsewhere; Unspecified |
| | hereditary and idiopathic peripheral neuropathy |
| 3 | Peroneal muscular atrophy; Abnormality of gait; Hereditary |
| | peripheral neuropathy; Other extrapyramidal diseases and abnormal |
| | movement disorders; Acquired foot deformity |

### 7.7 Bipolar Disorder

Bipolar Disorder or “Manic-depressive illness” causes extreme mood shifts including emotional highs (mania or hypomania) and lows (depression). About 2.6% of the population (5.7 million American adults) suffers from this disorder in any given year.

Individuals in the population were determined to be affecteds based for Bipolar Disorder and unipolar disorder. The ICD 10 code of Bipolar Disorder is F31; ICD 9 codes are 296.4 to 296.7. A subset (35 to 40%) of patients receives Lithium prescription. The ICD 10 code of Unipolar/Major depressive disorder is F32, F33, F39; ICD-9 codes are 296.2/.3/.9 (Secondary within a family network). Individuals with autism (ICD-10 code F84) and mental retardation (ICD-10 codes F70.9, F71.9, F72.9, F73.9, F79.9) were excluded from the affected set. The prevalence of the binary traits, in the cohort, for Bipolar Disorder (F319—3.2%) and unipolar disorders (F31, F32, and F33—0.0%, 4.1% and 2.1%, respectively) were under 5%.

A pedigree enriched for binary trait for Bipolar Disorder was isolated from the first degree family network.

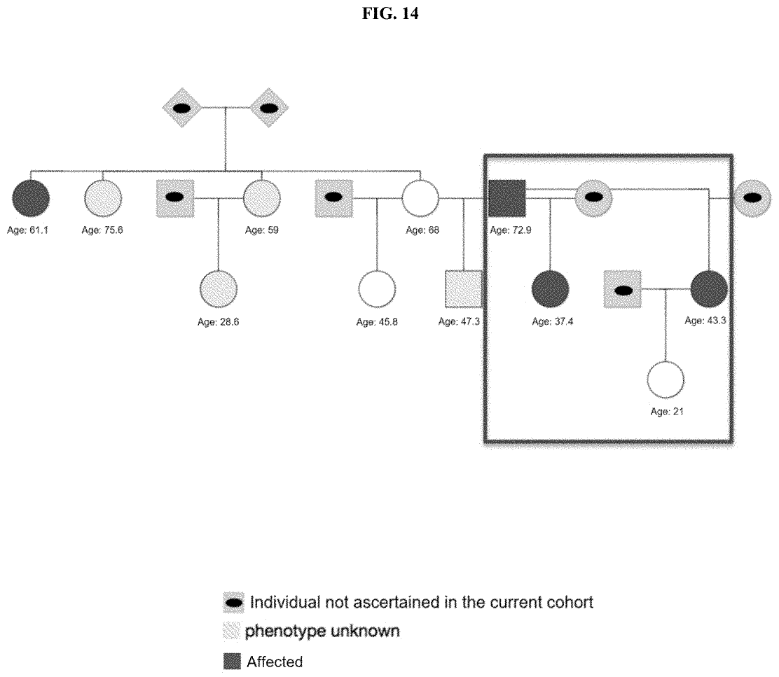

The first-degree pedigree was evaluated to ensure that it had only one possible structure and had at least three affecteds with a common ancestor (See FIG. 14). The segregation analysis performed on the enriched pedigree generated a list of possible variants co-segregating with the phenotype (Table 13). The variant C20orf203 co-segregating with the phenotype is deleterious and non-conserved.

TABLE 13

| GENE | GENE NAME | NT_CHANGE | AA_CHANGE | TGP_FREQ | EXAC_FREQ |
|---|---|---|---|---|---|
| C20orf203 | chromosome | c.113delC | p.T38fs | na | na |
| | 20 open | | | | |
| | reading | | | | |
| | frame 203 | | | | |
| TUBGCP6 | tubulin | c.C2119T | p.Q707X | na | na |
| | gamma | | | | |
| | complex | | | | |
| | associated | | | | |
| | protein 6 | | | | |
| SEC14L3 | SEC14 | c.A346T | p.K116X | na | 8.38E−06 |
| | like lipid | | | | |
| | binding 3 | | | | |
| TMPRSS4 | transmembrane | c.C358T | p.Q120X | na | 8.27E−06 |
| | protease, | | | | |
| | serine 4 | | | | |
| CARD10 | caspase | c.C550A | p.R184S | na | na |
| | recruitment | | | | |
| | domain | | | | |
| | family | | | | |
| | member 10 | | | | |
| LMAN1L | lectin, | c.C568T | p.R190W | na | 4.79E−05 |
| | mannose | | | | |
| | binding 1 | | | | |
| | like | | | | |
| C10orf71 | chromosome | c.C3715A | p.P2139T | na | na |
| | 10 open | | | | |
| | reading | | | | |
| | frame 71 | | | | |
| TMEM181 | transmembrane | c.G1657A | p.A553T | na | na |
| | protein 181 | | | | |
| MAN2B1 | mannosidase | c.G2094C | p.Q698H | na | 4.94E−05 |
| | alpha | | | | |
| | class 2B | | | | |
| | member 1 | | | | |
| IFT172 | intraflagellar | c.G2681A | p.R894H | na | na |
| | transport 172 | | | | |
| PIWIL1 | piwi like | c.G152A | p.R51Q | na | 8.26E−06 |
| | RNA- | | | | |
| | mediated | | | | |
| | gene | | | | |
| | silencing 1 | | | | |
| ACVRL1 | activing A | c.G948C | p.E316D | na | 4.16E−05 |
| | receptor | | | | |
| | like type 1 | | | | |
| TTLL10 | tubulin | c.G211A | p.E71K | 0.00002 | 3.37E−05 |
| | tyrosine | | | | |
| | ligase like | | | | |
| | 10 | | | | |
| CDBFA2T2 | CBFA2/ | c.C135A | p.N45K | na | 8.24E−06 |
| | RUNX1 | | | | |
| | translocation | | | | |
| | partner 2 | | | | |
| ZBTB2 | zinc finger | c.A1493G | p.H498R | na | 1.65E−05 |
| | and BTB | | | | |
| | domain | | | | |
| | containing 2 | | | | |

| GENE | RGC_FREQ | CONSERVATION | PREDICTION | GENOTYPES |
|---|---|---|---|---|
| C20orf203 | 1.11E−05 | na | deleterious | HET = 4/HOM = 0 |
| TUBGCP6 | 1.11E−05 | conserved | deleterious | HET = 4/HOM = 0 |
| SEC14L3 | 1.11E−05 | conserved | deleterious | HET = 4/HOM = 0 |
| TMPRSS4 | 2.21E−05 | conserved | tolerated | HET = 8/HOM = 0 |
| CARD10 | 3.88E−05 | conserved | deleterious | HET = 14/HOM = 0 |
| LMAN1L | 0.000144 | conserved | tolerated | HET = 52/HOM = 0 |
| C10orf71 | 1.38E−05 | conserved | deleterious | HET = 5/HOM = 0 |
| TMEM181 | 1.38E−05 | conserved | tolerated | HET = 5/HOM = 0 |
| MAN2B1 | 7.75E−05 | neutral | deleterious | HET = 28/HOM = 0 |
| IFT172 | 3.05E−05 | conserved | deleterious | HET = 11/HOM = 0 |
| PIWIL1 | 3.05E−05 | conserved | deleterious | HET = 11/HOM = 0 |
| ACVRL1 | 0.000125 | neutral | deleterious | HET = 45/HOM = 0 |
| TTLL10 | 1.66E−05 | neutral | tolerated | HET = 6/HOM = 0 |
| CDBFA2T2 | 0.000105 | conserved | deleterious | HET = 38/HOM = 0 |
| ZBTB2 | 3.05E−05 | conserved | tolerated | HET = 11/HOM = 0 |

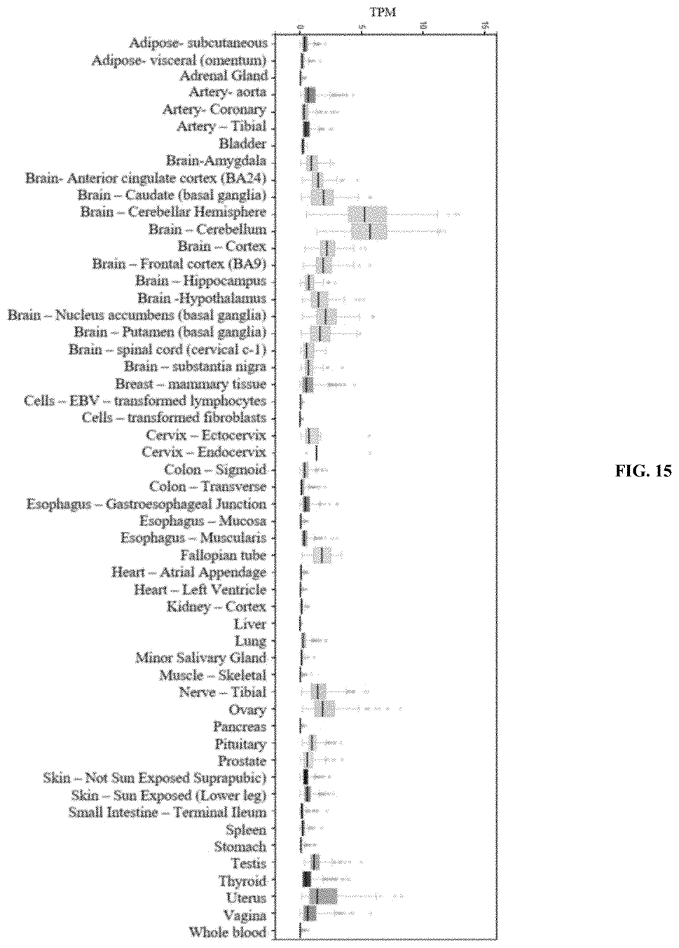

FLJ33706 (alternative gene symbol C20orf203) has been identified as the possible variant responsible for nicotine addiction. The gene expression data of transcripts per million (TPM) of chromosome 20 open reading frame 203 (C20orf203) encoded in various tissues, but primarily expressed in the cerebellar hemisphere and the cerebellum of the brain (FIG. 15). Linkage studies have identified rs17123507, an SNP located in the 3′UTR of FLJ33706, as significantly associated with susceptibility to nicotine addiction (Li et al. PLoS Computational Biology (2010) 6: e1000734).

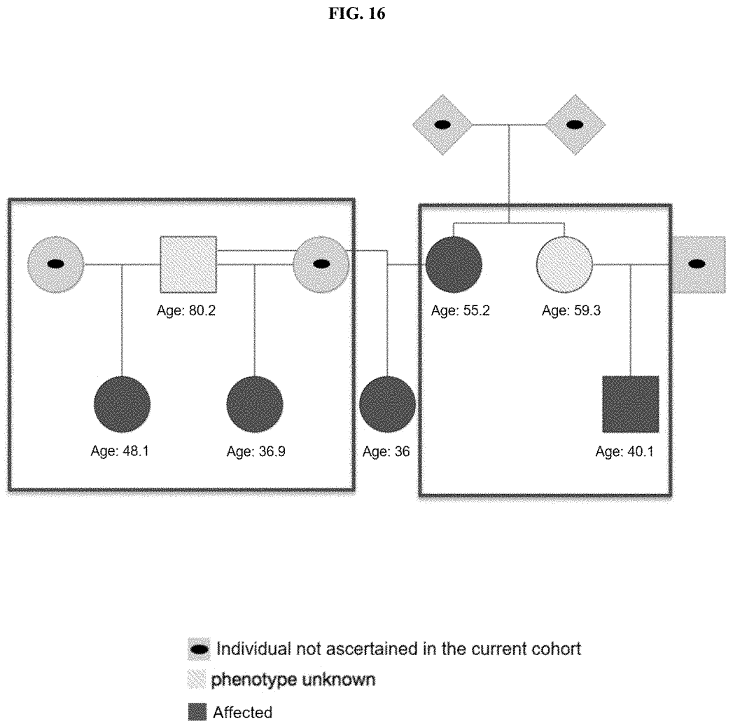

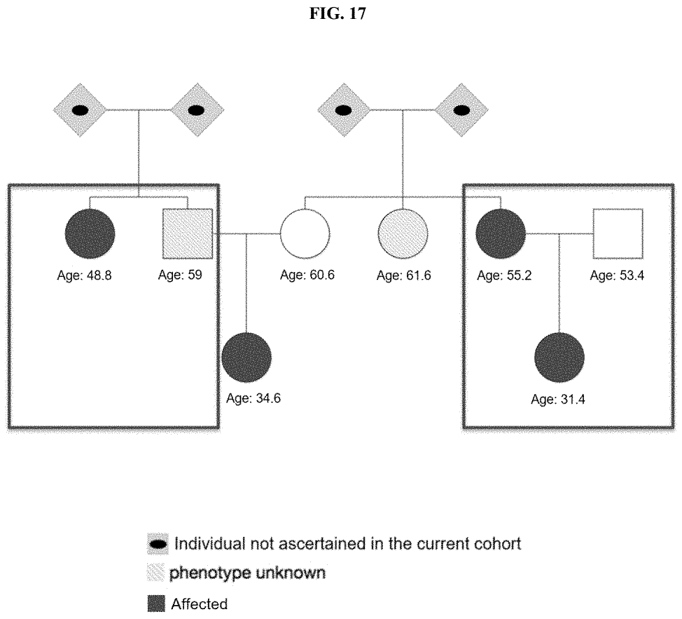

Further, two more enriched pedigrees were identified (See FIGS. 16 and 17; Table 14 and 15). Both the pedigrees had only one possible structure and had more than three affecteds with a common ancestor.

TABLE 14

| | | maximum | maximum | | |
|---|---|---|---|---|---|
| | | affecteds | informative | number of | |
| | | with | affecteds | affected | |
| Serial | number of | common | with common | sibling | pedigree |
| No. | affecteds | ancestor | ancestor | pairs | generations |
| 1 | 5 | 3 | 3 | 0 | 2 |
| 2 | 4 | 3 | 2 | 0 | 2 |

TABLE 15

| | | | | | number of | |
|---|---|---|---|---|---|---|
| | | | number of | number of | age consistent | |
| | affected | number of | unknown | samples in | possible | mode of |
| Serial No. | generations | unaffecteds | affecteds | pedigree | pedigrees | inheritance |
| 1 | 2 | 2 | 0 | 7 | 1 | dominant |
| 2 | 2 | 3 | 1 | 8 | 1 | dominant |

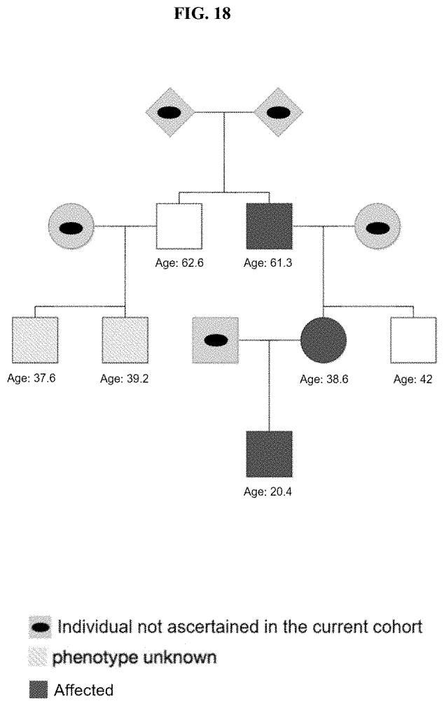

Additionally, another pedigree enriched for the binary trait for Bipolar Disorder had only one possible structure and had more than three affecteds with a common ancestor (See FIG. 18).

The variant analysis performed on the enriched pedigree generated a list of possible variants co-segregating with the phenotype (Table 16).

TABLE 16

| GENE | GENE NAME | NT CHANGE | AA CHANGE | TGP FREQ | EXAC FREQ |
|---|---|---|---|---|---|
| MCPH1 | microcephalin 1 | c.2453-1G > C) | | na | 1.675E−05 |
| NOL8 | nucleolar | c.38delG | p.G13fs | na | 166E−05 |
| | protein | | | | |
| INSM2 | INSM | c.1031_1035del | p.344_345del | na | na |
| | transcriptional | | | | |
| | repressor 2 | | | | |
| CDRT15L2 | CMT1A | c.345_346del | p.115_1116del | na | 0.0002 |
| | duplicated | | | | |
| | transcripts | | | | |
| | 15-like 2 | | | | |
| SHPK | sedoheptulokinase | c.C355T | p.R119X | 0.000599 | 0.0005 |
| INSM2 | INSM | c.A686G | p.D229G | na | 5.56E−05 |
| | transcriptional | | | | |
| | repressor 2 | | | | |
| DAAM1 | dishevelled | c.T2474G | p.F825C | na | 6.59E−05 |
| | associated | | | | |
| | activator of | | | | |
| | morphogenesis | | | | |
| | 1 | | | | |
| APBA2 | amyloid | c.C1417T | p.R473C | 0.0002 | 8.46E−06 |
| | beta | | | | |
| | precursor | | | | |
| | protein | | | | |
| | binding | | | | |
| | family A | | | | |
| | member 2 | | | | |
| FAM107B | family | c.A155G | p.E52G | na | na |
| | with | | | | |
| | sequences | | | | |
| | similarity | | | | |
| | 107 | | | | |
| | member B | | | | |
| AKR1C3 | aldo-keto | c.G512T | p.R171M | na | 8.24E−06 |
| | reductase | | | | |
| | family 1 | | | | |
| | member | | | | |
| | C3 | | | | |
| PLEKHG3 | pleckstrin | c.G80T | p.G27V | na | 1.87E−05 |
| | homology | | | | |
| | and | | | | |
| | RhoGEF | | | | |
| | domain | | | | |
| | containing | | | | |
| | G3 | | | | |
| PDSS1 | prenyl | c.G1138A | p.A380T | na | 4.95E−05 |
| | (decaprenyl) | | | | |
| | diphosphate | | | | |
| | synthase, | | | | |
| | subunit 1 | | | | |
| CCM2 | CCM2 | c.G391A | p.D131N | 0.0002 | 0.0003 |
| | scaffolding | | | | |
| | protein | | | | |
| LRRC37B | leucine | c.A2188C | p.K730Q | na | 4.98E−05 |
| | rich repeat | | | | |
| | containing | | | | |
| | 37B | | | | |
| EML4 | echinoderm | c.A736T | p.N246Y | na | 1.65E−05 |
| | microtubule | | | | |
| | associated | | | | |
| | protein | | | | |
| | like 4 | | | | |
| ARHGEF10 | Rho guanine | c.C3097T | p.H1033Y | na | na |
| | nucleotide | | | | |
| | exchange | | | | |
| | factor 10 | | | | |
| STAT5A | signal | c.G779A | p.R260Q | na | 3.34E−05 |
| | transducer | | | | |
| | and | | | | |
| | activator of | | | | |
| | transcription | | | | |
| | 5A | | | | |
| RP1L1 | retinitis | c.G1493T | p.S4981 | na | 8.76E−06 |
| | pigmentosa- | | | | |
| | 1-like 1 | | | | |
| ATAD2 | ATPase | c.C2219A | p.S740Y | na | na |
| | family, | | | | |
| | AAA | | | | |
| | domain | | | | |
| | containing 2 | | | | |
| RBM43 | RNA | c.C35G | p.A12G | na | 3.33E−05 |
| | binding | | | | |
| | motif | | | | |
| | protein 43 | | | | |

| GENE | RGC FREQ | CONSERVATION | PREDICTION | GENOTYPES |
|---|---|---|---|---|
| MCPH1 | 3.32E−05 | conserved | deleterious | HET = 12/HOM = 0 |
| NOL8 | 3.60E−05 | na | deleterious | HET = 13/HOM = 0 |
| INSM2 | 5.54E−05 | na | deleterious | HET = 20/HOM = 0 |
| CDRT15L2 | 0.000235 | na | deleterious | HET = 85/HOM = 0 |
| SHPK | 0.000684 | neutral | tolerated | HET = 245/H0M = 0 |
| INSM2 | 0.00013 | conserved | deleterious | HET = 47/HOM = 0 |
| DAAM1 | 0.000191 | conserved | deleterious | HET = 69/HOM = 0 |
| APBA2 | 9.97E−05 | conserved | deleterious | HET = 36/HOM = 0 |
| FAM107B | 5.26E−05 | conserved | deleterious | HET = 19/HOM = 0 |
| AKR1C3 | 6.64E−05 | neutral | deleterious | HET = 24/HOM = 0 |
| PLEKHG3 | 3.05E−05 | conserved | deleterious | HET = 11/HOM = 0 |
| PDSS1 | 8.30E−05 | conserved | deleterious | HET = 30/HOM = 0 |
| CCM2 | 0.000221 | conserved | deleterious | HET = 80/HOM = 0 |
| LRRC37B | 9.41E−05 | neutral | tolerated | HET = 34/HOM = 0 |
| EML4 | 3.88E−05 | conserved | deleterious | HET = 14/HOM = 0 |
| ARHGEF10 | 1.66E−05 | conserved | deleterious | HET = 6/HOM = 0 |
| STAT5A | 0.000188 | conserved | deleterious | HET = 66/HOM = 1 |
| RP1L1 | 4.43E−05 | neutral | tolerated | HET = 16/HOM = 0 |
| ATAD2 | 0.000102 | conserved | tolerated | HET = 37/HOM = 0 |
| RBM43 | 0.000147 | neutral | tolerated | HET = 53/HOM = 0 |

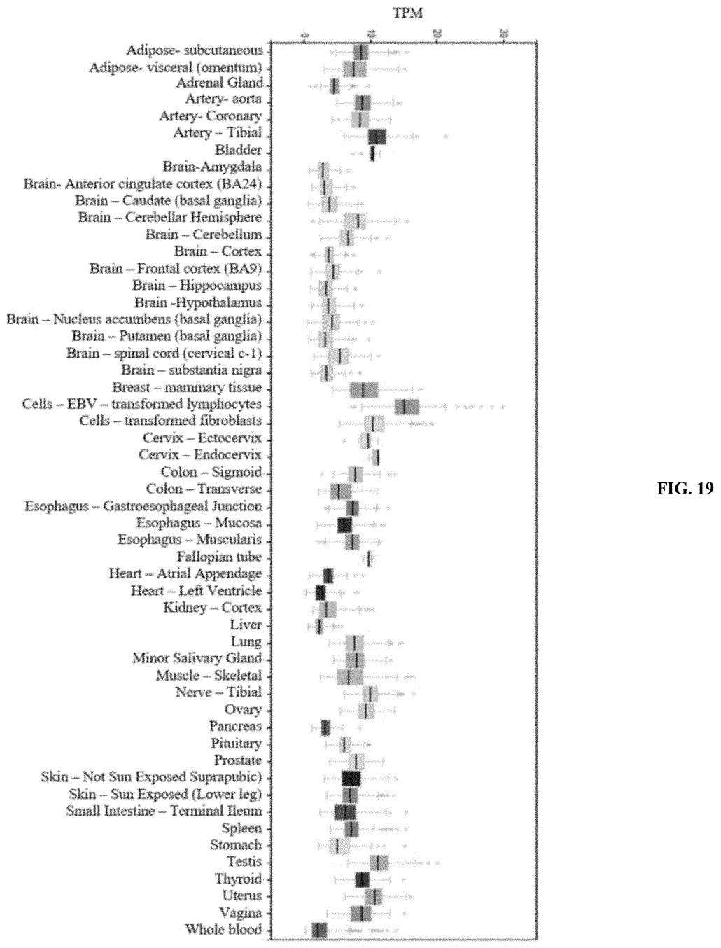

Among the listed variants in Table 17, microcephalin 1 (MCPH1) is a reported pathogenic variant for primary microcephaly. The gene expression data of transcripts per million (TPM) of MCPH1 encoded in various tissues indicated a high occurrence in several tissues (See FIG. 19)

Primary microcephaly type 1 is characterized by head circumference more than 3 standard deviations below the age-related mean. Brain weight is markedly reduced and the cerebral cortex is disproportionately small. Affected individuals have severe intellectual disability. Some MCHP1 patients also present growth retardation, short stature, and misregulated chromosome condensation as indicated by a high number of prophase-like cells detected in cytogenetic preparations and poor-quality metaphase G-banding.

TABLE 17

| GENE | GENE_NAME | NT_CHANGE | AA_CHANGE | TGP_FREQ | EXAC_FREQ |
|---|---|---|---|---|---|
| MCPH1 | microcephalin 1 | c.2453-1G > C | splicing | na | 0.00001667 |

| GENE | RGC_FREQ | CONSERV | PREDICTION | GENOTYPES |
|---|---|---|---|---|
| MCPH1 | 0.00003321 | conserved | deleterious | HET = 12/HOM = 0 |

### 7.8 Thalassemia

Thalassemia is an inherited blood disorder characterized by less hemoglobin and fewer red blood cells in your body than normal. The low hemoglobin and fewer red blood cells of thalassemia may cause anemia, leaving a patient fatigued.

The ICD 10 code of thalassemia is D56.

A pedigree enriched for binary trait for thalassemia was isolated from the first degree family network.

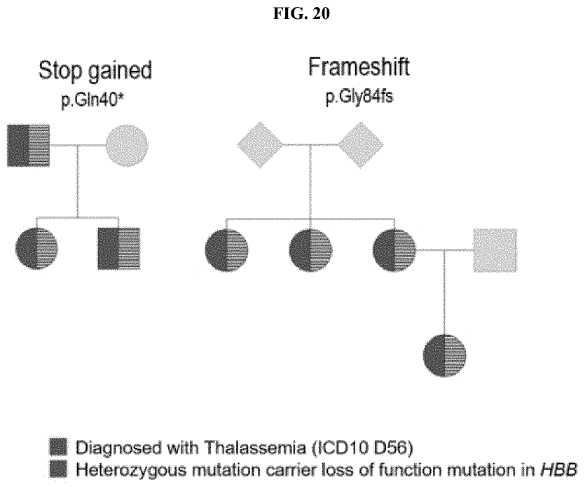

The first-degree pedigree was evaluated to ensure that it had only one possible structure and had at least three affecteds with a common ancestor (See FIG. 20). Two enriched pedigrees were identified (See FIG. 20). Both the pedigrees had only one possible structure and had three or more affecteds.

The variant analysis performed on the enriched pedigrees generated a list of possible variants of the HBB gene co-segregating with the phenotype. The HBB gene provides instructions for making a protein called beta-globin. Beta-globin is a component (subunit) of a larger protein called hemoglobin, which is located inside red blood cells. In adults, hemoglobin normally consists of four protein subunits: two subunits of beta-globin and two subunits of another protein called alpha-globin, which is produced from another gene called HBA. Each of these protein subunits is attached (bound) to an iron-containing molecule called heme; each heme contains an iron molecule in its center that can bind to one oxygen molecule. Hemoglobin within red blood cells binds to oxygen molecules in the lungs. These cells then travel through the bloodstream and deliver oxygen to tissues throughout the body. The diseases associated with the HBB gene include Beta-Thalassemia and Sickle Cell Anemia.

The two mutations identified in the HBB gene co-segregating with the phenotype were stop gain mutation at Gln40 and a frameshift mutation at Gly84 (association analysis p-value is <3.1×10⁻¹⁹). These identified mutations can be studied and possible therapeutic approaches to treat familial thalassemia can be further developed using this knowledge.

### 7.10 Decreased Alkaline Phosphatase Outpatient Central Tendency Value

Routine laboratory testing for Alkaline Phosphatase is performed quite frequently in the hospital for both diagnostic purposes in symptomatic patients as well as for screening purposes in asymptomatic patients. Although Alkaline Phosphatase enzyme is present in tissues throughout the body, it is most often elevated in patients with liver and bone disease.

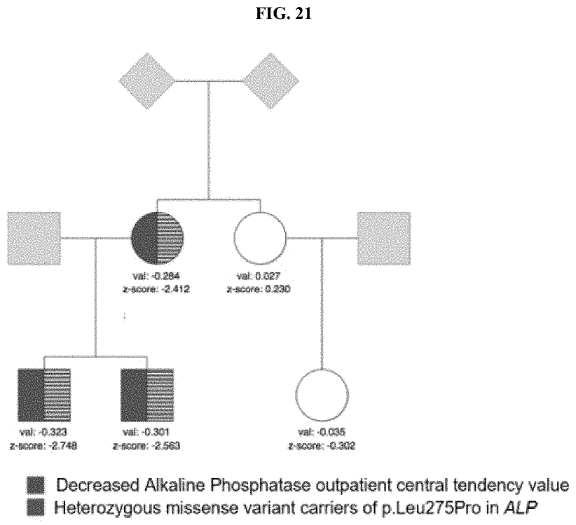

A pedigree enriched for decreased Alkaline Phosphatase levels was created and was evaluated to ensure that it had only one possible structure and had at least three affecteds with a common ancestor (See FIG. 21).

A variant analysis performed on the enriched pedigree indicated that a missense mutation in the ALPL gene co-segregated with the phenotype. The ALPL gene provides instructions for making an enzyme called tissue-nonspecific alkaline phosphatase (TNSALP). This enzyme plays an important role in the growth and development of bones and teeth. It is also active in many other tissues, particularly in the liver and kidneys. This enzyme acts as a phosphatase, which means that it removes clusters of oxygen and phosphorus atoms (phosphate groups) from other molecules. TNSALP is essential for the process of mineralization, in which minerals such as calcium and phosphorus are deposited in developing bones and teeth. Mineralization is critical for the formation of bones that are strong and rigid and teeth that can withstand chewing and grinding. The heterozygous missense mutation identified in the ALPL gene was at Leu275 (Leu275Pro) (See FIG. 21) (association analysis p-value is <7.2×10⁻²⁷). The association results and mendelian segregation provide a somewhat independent evidence of the association between the variant and the decrease in the quantitative trait.

What is claimed is:

1. A system for identifying disease causing variant, the system comprising:
a data processor and a memory coupled with the data processor, the processor being configured to:
generate a first degree network of individuals based on sequencing data of a cohort;
identify whether an individual in the first degree network as an affected or an unaffected, wherein the individual with at least one binary trait, extreme quantitative trait, or combination thereof is identified as affected and the individual without the at least one binary trait or the extreme quantitative trait is identified as unaffected; and
generate at least one enriched pedigree containing the individuals including designation as affected or unaffected.

2. The system of claim 1, wherein the processor is further configured to perform segregation analysis to identify variant trait pairs that co-segregate within and across the at least one enriched pedigree.

3. The system of claim 1, wherein the processor is further configured to perform segregation analysis to analyze the variant trait pairs to determine the disease-causing variant.

4. The system of claim 1, wherein the sequencing data can be selected from the group consisting of exome sequencing data or whole genome sequencing data or genotype array data.

5. The system of claim 1, wherein the processor is further configured to identify the identified affected as unaffected if a prevalence of the at least one binary trait in the cohort is over 5%.

6. The system of claim 1, wherein the processor is further configured to identify the identified affected as unaffected if the at least one extreme quantitative trait of the individual is less than two standard deviations from a mean quantitative trait of the cohort.

7. The system of claim 1, wherein the enriched pedigree generated by the system has a single possible structure.

8. The system of claim 1, wherein the enriched pedigree generated by the system includes three or more affecteds with a common ancestor.

9. The system of claim 1, wherein the enriched pedigree generated by the system includes one or more affecteds having parents determined to be unaffected.

10. The system of claim 1, wherein the processor is further configured to prioritize the at least one pedigree to perform the segregation analysis.

11. The system of claim 10, wherein the prioritizing the at least one pedigree comprises selecting the at least one pedigree including at least one related unaffected.

12. The system of claim 10, wherein the prioritizing the at least one pedigree comprises selecting the at least one pedigree including at least two affected siblings.

13. The system of claim 10, wherein the segregation analysis forms a dominant genetic model of segregation.

14. The method of claim 10, wherein the segregation analysis forms a recessive genetic model of segregation.

15. The method of claim 3, wherein analyzing the variant trait pairs to determine the disease-causing variant is carried out using family-based association analysis.

16. A method of identifying a disease-causing variant, comprising:
generating a first degree network of individuals based on sequencing data of a cohort;
identifying an individual in the first degree network as an affected or an unaffected, wherein the individual affected by at least one binary trait, extreme quantitative trait, or combination thereof is identified as an affected and the individual that is not affected by at least one binary trait or the extreme quantitative trait is identified as an unaffected;
creating at least one enriched pedigree containing the individuals including designation as affected or unaffected;
performing segregation analysis to identify variant trait pairs that co-segregate within and across the at least one enriched pedigree; and
analyzing the variant trait pairs to identify the disease-causing variant.

17. A non-transitory computer readable medium storing instructions for causing a processor to perform a method for identifying a disease-causing variant, comprising:
generating a first degree network of individuals based on exome sequencing data of a cohort;
identifying an individual in the first degree network as an affected or an unaffected, wherein the individual with at least one binary trait, extreme quantitative trait, or combination thereof is identified as an affected and the individual without the at least one binary trait or the extreme quantitative trait is identified as an unaffected;
creating at least one enriched pedigree containing the individuals including designation as affected or unaffected;
performing segregation analysis to identify variant trait pairs that co-segregate within and across the at least one enriched pedigree; and
analyzing the variant trait pairs to determine the disease-causing variant.

18. A system for generating an enriched pedigree, the system comprising:
a data processor and a memory coupled with the data processor, the processor being configured to:
generate a first degree network of individuals based on sequencing data of a cohort;
identify whether an individual in the first degree network as an affected or an unaffected, wherein the individual with at least one binary trait, extreme quantitative trait, or combination thereof is identified as affected and the individual without the at least one binary trait or the extreme quantitative trait is identified as unaffected; and
generate at least one enriched pedigree containing the individuals including designation as affected or unaffected.
